(19) United States
(12) Patent Application Publication
(10) Pub. No.: US 2023/0303549 A1
(43) Pub. Date: Sep. 28, 2023

(54) SUBSTITUTED HETEROARYLOXYPYRIDINES, THE SALTS THEREOF AND THEIR USE AS HERBICIDAL AGENTS

(71) Applicant: Bayer Aktiengesellschaft, Leverkusen (DE)

(72) Inventors: Michael Charles MCLEOD, Frankfurt (DE); Ralf BRAUN, Ramberg (DE); Harald JAKOBI, Frankfurt (DE); Stefan SCHNATTERER, Hattersheim (DE); Catherine Simone MEISTER, Ivanhoe, Victoria (AU); Dirk SCHMUTZLER, Hattersheim (DE); Anu Bheemaiah MACHETTIRA, Frankfurt am Main (DE); Elisabeth ASMUS, Hoesbach (DE); Christopher Hugh ROSINGER, Hofheim (DE); Elmar GATZWEILER, Bad Nauheim (DE); Anna Maria REINGRUBER, Heppenheim (DE); Jan DITTGEN, Frankfurt (DE); Birgit BOLLENBACH-WAHL, Weiler/Bingen (DE)

(21) Appl. No.: 18/003,745

(22) PCT Filed: Jun. 28, 2021
(86) PCT No.: PCT/EP2021/067667

§ 371 Date: Dec. 29, 2022

(30) Foreign Application Priority Data
Jun. 30, 2020 (EP) 20183158.3

(51) Int. Cl. C07D 413/14; A01N 25/32; A01N 43/80; C07D 413/04; C07D 401/14; C07D 401/04; A01N 43/56; A01P 13/00

(52) U.S. Cl. CPC C07D 413/14; A01N 25/32; A01N 43/80; C07D 413/04; C07D 401/14; C07D 401/04; A01N 43/56; A01P 13/00

(57) ABSTRACT

The invention relates to substituted heteroaryloxypyridines of the general formula (I) and (I) to their use as herbicides, in particular for controlling weeds and/or weed grasses in crops of cultivated plants and/or as plant growth regulators for influencing the growth of crops of cultivated plants. The present invention also relates to herbicidal and/or plant growth-controlling agents comprising one or more compounds of the general formula (I).

The invention relates to the technical field of crop protection products, in particular that of herbicides for selective control of broad-leaved weeds and weed grasses in crops of useful plants.

Specifically, the present invention relates to substituted heteroaryloxypyridines and salts thereof, to processes for preparation thereof and to the use thereof as herbicides.

In their application, crop protection products known to date for the selective control of harmful plants in crops of useful plants or active ingredients for controlling unwanted vegetation sometimes have disadvantages, whether (a) that they have insufficient herbicidal activity, if any, against particular harmful plants, (b) that the spectrum of harmful plants which can be controlled with an active ingredient is not wide enough, (c) that their selectivity in crops of useful plants is too low and/or (d) that they have a toxicologically unfavorable profile. Furthermore, some active ingredients which can be used as plant growth regulators for a number of useful plants cause undesirably reduced harvest yields in other useful plants or are compatible with the crop plant only within a narrow application rate range, if at all. Some of the known active ingredients cannot be produced economically on an industrial scale owing to precursors and reagents which are difficult to obtain, or they have only insufficient chemical stabilities. In the case of other active ingredients, the activity is too highly dependent on environmental conditions, such as weather and soil conditions.

The herbicidal action of these known compounds, especially at low application rates, and/or the compatibility thereof with crop plants is still in need of improvement.

WO 2016/149315 describes various 3-pyrimidyloxypyridines as herbicides.

WO 2020/002089 describes various 2-heteroaryloxypyridines having herbicidal action that bear a ring bonded to the pyridine via a 1-atom bridge as substituent in the 3 position of the pyridine.

WO 2004/035564 describes various 2-heteroaryloxypyridines as herbicides that are substituted by pyrazolyl radicals in the 3 position of the pyridine. By contrast, there is still no description of 2-heteroaryloxypyridines substituted by other 5-membered heterocyclic rings in the 3 position of the pyridine, and salts thereof.

It has now been found that, surprisingly, particular 2-heteroaryloxypyridines and/or salts thereof are of particularly good suitability as active herbicidal ingredients.

The present invention thus provides substituted heteroaryloxypyridines of the general formula (I) or salts thereof

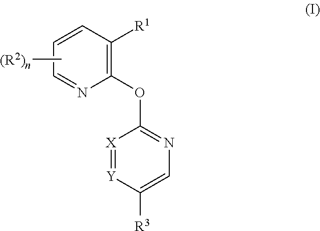

in which
R¹ is an optionally substituted 5-membered heteroaryl ring optionally substituted by up to 3 substituents independently selected from the group of IV,
R² is independently halogen, hydroxy, amino, cyano, nitro, formyl, formamide, (C₁-C₄)-alkyl, (C₁-C₄)-haloalkyl, (C₃-C₆)-cycloalkyl, (C₂-C₄)-alkenyl, (C₂-C₄)-alkynyl, (C₂-C₄)-haloalkenyl, (C₂-C₄)-haloalkynyl, (C₁-C₄)-alkoxy, (C₁-C₄)-haloalkoxy, (C₃-C₆)-cycloalkoxy, (C₁-C₄)-alkenyloxy, (C₁-C₄)-alkynyloxy, (C₁-C₄)-alkylthio, (C₁-C₄)-haloalkylthio, (C₃-C₆)-cycloalkylthio, (C₁-C₄)-alkylsulfinyl, (C₁-C₄)-haloalkylsulfinyl, (C₃-C₆)-cycloalkylsulfinyl, (C₁-C₄)-alkylsulfonyl, (C₁-C₄)-haloalkylsulfonyl, (C₃-C₆)-cycloalkylsulfonyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-alkylsulfinyl-(C₁-C₄)-alkyl, (C₁-C₄)-alkylsulfonyl-(C (C₁-C₄)-alkylcarbonyl, (C -C4)-haloalkylcarbonyl, (C₃-C₆)-cycloalkylcarbonyl, carboxyl, (C₁-C₄)-alkoxycarbonyl, (C₁-C₄)-haloalkoxycarbonyl, (C₃-C₆)-cycloalkoxycarbonyl, (C₁-C₄)-alkylaminocarbonyl, (C₂-C₆)-dialkylaminocarbonyl, (C₃-C₆)-cycloalkylaminocarbonyl, (C₁-C₄)-alkylcarbonylamino, (C₁-C₄)-haloalkylcarbonylamino, (C₂-C₆)-cycloalkylcarbonylamino, (C₁-C₄)-alkoxycarbonylamino, (C₁-C₄)-alkylaminocarbonylamino, (C₂-C₆)-dialkylaminocarbonylamino, carboxy-(C₁-C₄)-alkyl, (C₁-C₄)-alkoxycarbonyl-(C₁-C₄)-alkyl, (C₁-C₄)-haloalkoxycarbonyl-(C₁-C₄)-alkyl, (C₃-C₆)-cycloalkoxycarbonyl-(C₁-C₄)-alkyl, (C₁-C₄)-alkylaminosulfonyl, (C₂-C₆)-dialkylaminosulfonyl or (C₃-C₆)-trialkylsilyl,
n is 0, 1, 2 or 3,
R³ is halogen, cyano, nitro, (C₁-C₄)-alkyl or (C₁-C₄)-haloalkyl,
R⁴ is hydrogen, halogen, hydroxy, amino, cyano, formyl, (C₁-C₄)-alkyl, (C₁-C₄)-haloalkyl, (C₃-C₆)-cycloalkyl, (C₁-C₄)-alkoxy, (C₁-C₄)-haloalkoxy, (C₁-C₄)-alkoxycarbonyl, (C₁-C₄)-alkoxythiocarbonyl, (C₃-C₆)-cycloalkyl-(C₁-C₄)-alkyl, (C₁-C₄)-alkyl-(C₃-C₆)-cycloalkyl or (C₁-C₄)-alkoxy-(C₁-C₄)-alkyl,
X is N or CR⁵,
Y is N or CH,
and
R⁵ is hydrogen, halogen or cyano, excluding compounds in which R¹ is an unsubstituted or substituted 3-pyrazole.

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. These salts then contain the conjugate base of the acid as anion. Suitable substituents in deprotonated form, for example sulfonic acids, particular sulfonamides or carboxylic acids, are capable of forming internal salts with groups, such as amino groups, which are themselves protonatable. Salts may also be formed by action of a base on compounds of the general formula (I). Suitable bases are, for example, organic amines such as trialkylamines, morpholine, piperidine and pyridine, and the hydroxides, carbonates and hydrogencarbonates of ammonium, alkali metals or alkaline earth metals, especially sodium hydroxide, potassium hydroxide, sodium carbonate, potassium carbonate, sodium hydrogencarbonate and potassium hydrogencarbonate. These salts are compounds in which the acidic hydrogen is replaced by an agriculturally suitable cation, for example metal salts, especially alkali metal salts or alkaline earth metal salts, in particular sodium and potassium salts, or else ammonium salts, salts with organic amines or quaternary ammonium salts, for example with cations of the formula [NR^{a}R^{b}R^{c}R^{d}]⁺ in which R^{a} to R^{d} are each independently an organic radical, especially alkyl, aryl, arylalkyl or alkylaryl. Also useful are alkylsulfonium and alkylsulfoxonium salts, such as (C₁-C₄)-trialkylsulfonium and (C₁-C₄)-trialkylsulfoxonium salts.

The heteroaryloxypyridines of the general formula (I) having substitution in accordance with the invention may, depending on external conditions such as pH, solvent and temperature, be present in various tautomeric structures, all of which are embraced by the general formula (I).

The compounds of the formula (I) used in accordance with the invention and salts thereof are referred to hereinafter as “compounds of the general formula (I)”.

The invention preferably provides compounds of the general formula (I) in which
R¹ is the groups R¹-1 to R¹-42:

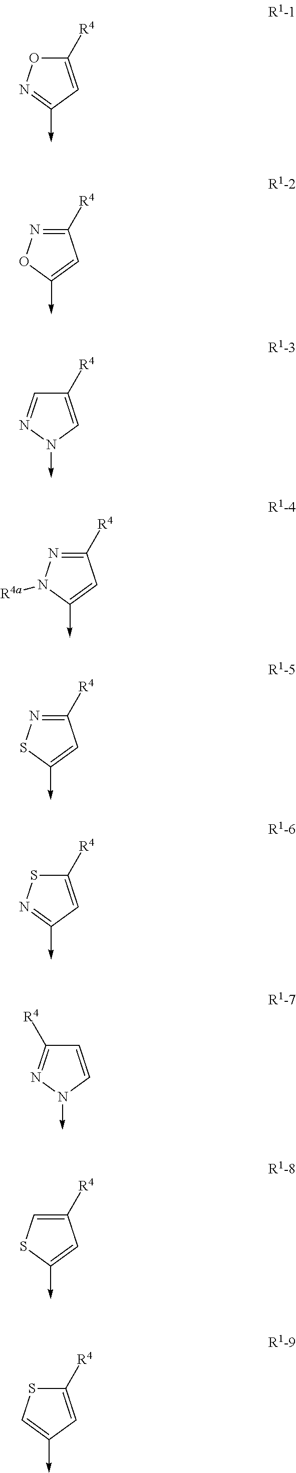

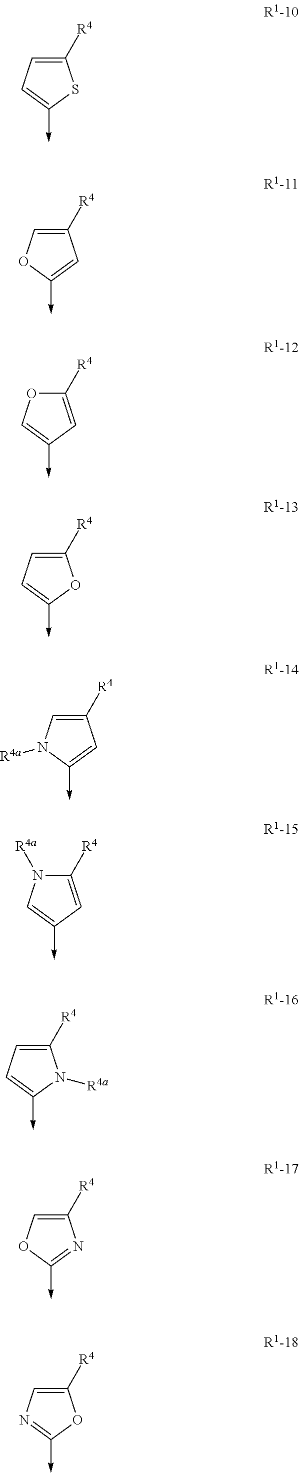

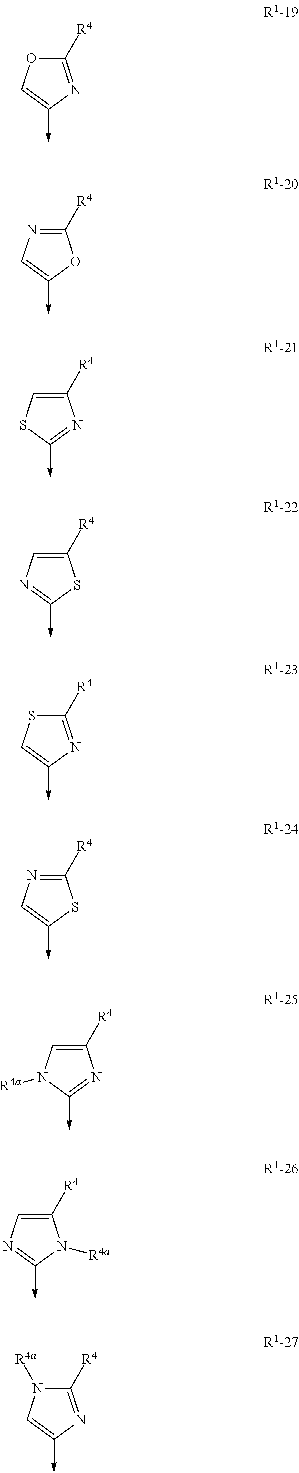

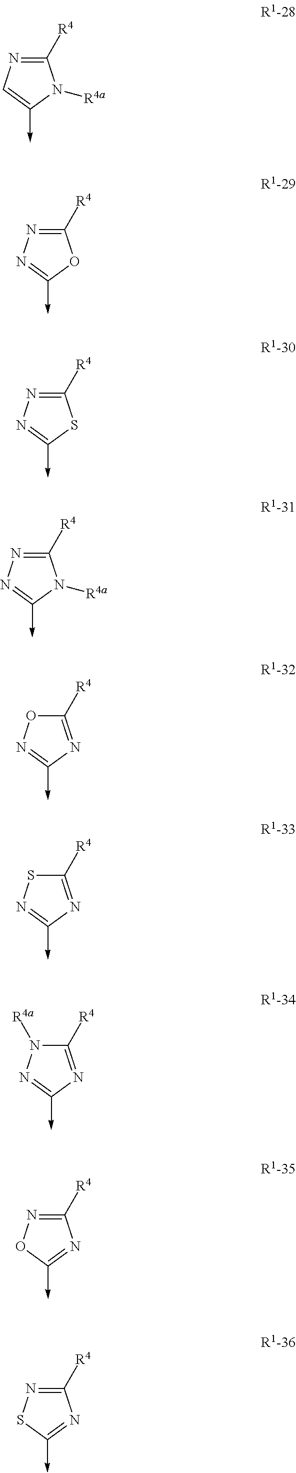

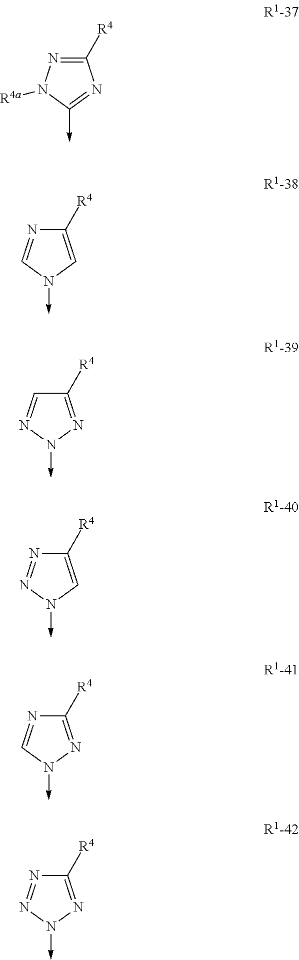

R² is independently halogen, hydroxy, amino, cyano, nitro, formyl, formamide, (C1-C4)-haloalkyl, (C₃-C₆)-cycloalkyl, (C₂-C₄)-alkenyl, (C₂-C₄)-alkynyl, (C₂-C₄)-haloalkenyl, (C₂-C₄)-haloalkynyl, (C₁-C₄)-alkoxy, (C₁-C₄)-haloalkoxy, (C₃-C₆)-cycloalkoxy, (C₁-C₄)-alkenyloxy, (C₁-C₄)-alkynyloxy, (C₁-C₄)-alkylthio, (C₁-C₄)-haloalkylthio, (C₃-C₆)-cycloalkylthio, (C₁-C₄)-alkylsulfinyl, (C₁-C₄)-haloalkylsulfinyl, (C₃-C₆)-cycloalkylsulfinyl, (C₁-C₄)-alkylsulfonyl, (C₁-C₄)-haloalkylsulfonyl, (C₃-C₆)-cycloalkylsulfonyl, (C₁-C₄)-alkoxy-(C₁-C₄)-alkyl, haloalkoxy-(C₁-C₄)-alkyl, (C₁-C₄)-alkylcarbonyl, (C₁-C₄)-haloalkylcarbonyl, (C₃-C₆)-cycloalkylcarbonyl, carboxyl, (C₁-C₄)-alkoxycarbonyl, (C₁-C₄)-haloalkoxycarbonyl, (C₃-C₆)-cycloalkoxycarbonyl, (C₁-C₄)-alkylaminocarbonyl, (C₂-C₆)-dialkylaminocarbonyl, (C₃-C₆)-cycloalkylaminocarbonyl, (C₁-C₄)-alkylcarbonylamino, (C₁-C₄)-haloalkylcarbonylamino, (C₂-C₆)-cycloalkylcarbonylamino, (C₁-C₄)-alkoxycarbonylamino or (C₃-C₆)-trialkylsilyl,
n is 0, 1 or 2,
R³ is halogen, cyano, nitro, (C₁-C₄)-alkyl or (C₁-C₄)-haloalkyl,
R⁴ is hydrogen, halogen, hydroxy, amino, cyano, formyl, (C₁-C₄)-haloalkyl, (C₃-C₆)-cycloalkyl, (C₁-C₄)-alkoxy, (C₁-C₄)-haloalkoxy, (C₁-C₄)-alkoxycarbonyl, (C₁-C₄)-alkoxythiocarbonyl, (C₃-C₆)-cycloalkyl-(C₁-C₄)-alkyl, (C₁-C₄)-alkyl-(C₃-C₆)-cycloalkyl or (C₁-C₄)-alkoxy-(C₁-C₄)-alkyl,
R^{4a} is hydrogen or (C₁-C₂)-alkyl,
X is N or CR⁵,
Y is N or CH,
and
R⁵ is hydrogen, halogen or cyano.

The invention more preferably provides compounds of the general formula (I) in which
R¹ is the groups R¹-1 to R¹-6,
R² is independently halogen, hydroxy, amino, cyano, nitro, (C₁-C₄)-alkyl, (C₁-C₄)-haloalkyl, (C₃-C₆)-cycloalkyl, (C₂-C₄)-alkenyl, (C₂-C₄)-alkynyl, (C₁-C₄)-alkoxy, (C₁-C₄)-haloalkoxy, (C₁-C₄)-alkylthio or (C₁-C₄)-alkylcarbonylamino,
n is 0, 1 or 2,
R³ is halogen, cyano, nitro, (C₁-C₂)-alkyl or (C₁-C₂)-haloalkyl,
R⁴ is hydrogen, halogen, hydroxy, amino, cyano, formyl, (C₁-C₄)-alkyl, (C₁-C₄)-haloalkyl, (C₃-C₆)-cycloalkyl, (C₁-C₄)-alkoxy, (C₁-C₄)-haloalkoxy, (C₁-C₄)-alkoxycarbonyl, (C₁-C₄)-alkoxythiocarbonyl, (C₃-C₆)-cycloalkyl-(C₁-C₄)-alkyl, (C₁-C₄)-alkyl-(C₃-C₆)-cycloalkyl or (C₁-C₄)-alkoxy-(C₁-C₄)-alkyl,
R^{4a} is hydrogen or (C₁-C₂)-alkyl,
X is N or CR⁵,
Y is N or CH,
and
R⁵ is hydrogen, halogen or cyano.

The invention very particularly preferably provides compounds of the general formula (I) in which
R¹ is the groups R¹-1 to R¹-3,
R² is independently halogen, amino, cyano, (C₁-C₂)-alkyl, (C₁-C₂)-haloalkyl or vinyl,
n is 0, 1 or 2,
R³ is halogen, cyano, nitro, (C₁-C₂)-alkyl or (C₁-C₂)-haloalkyl,
R⁴ is hydrogen, halogen, cyano, formyl, (C₁-C₂)-alkyl, (C₁-C₂)-haloalkyl, (C₃-C₆)-cycloalkyl, (C₁-C₂)-alkoxy, (C₁-C₂)-haloalkoxy, (C₁-C₄)-alkoxycarbonyl, (C₁-C₄)-alkoxythiocarbonyl, (C₃-C₆)-cycloalkyl-(C₁-C₂)-alkyl, (C₁-C₂)-alkyl-(C₃-C₆)-cycloalkyl or (C₁-C₂)-alkoxy-(C₁-C₂)-alkyl,
X is N or CR⁵,
Y is N or CH,
and
R⁵ is hydrogen, halogen or cyano.

The invention more preferably further provides compounds of the general formula (I) in which

R¹ is the groups R¹-1 to R¹-3,
R² is independently chlorine, bromine, cyano or methyl,
n is 1 or 2,
R³ is fluorine, chlorine, bromine, cyano, nitro or trifluoromethyl,
R⁴ is hydrogen, chlorine, bromine, iodine, trifluoromethyl, difluoromethyl, chlorofluoromethyl, difluorochloromethyl, dichloromethyl, trichloromethyl, difluorobromomethyl, cyclopropyl, cyclopropylmethyl, (1-methyl)cyclopropyl or methoxymethyl,
X is N or CR⁵,
Y is N or CH,
and
R⁵ is hydrogen, fluorine, chlorine or cyano.

The invention most preferably provides compounds of the general formula (I) in which
R¹ is the groups R¹-1, R¹-2 and R¹-3,
R² is methyl,
n is 1 or 2,
R³ is fluorine, chlorine, bromine, cyano or trifluoromethyl,
R⁴ is hydrogen, bromine, iodine, cyclopropyl, trifluoromethyl, difluoromethyl, chlorofluoromethyl, difluorochloromethyl, cyclopropylmethyl or (1-methyl)cyclopropyl,
X is N or CR⁵,
Y is N or CH,
and
R⁵ is hydrogen, fluorine, chlorine or cyano. The definitions of radicals listed above in general terms or within areas of preference apply both to the end products of the general formula (I) and correspondingly to the starting materials or intermediates required for preparation in each case. These radical definitions can be combined with one another as desired, i.e. including combinations between the given preferred ranges.

Of particular interest, primarily for reasons of higher herbicidal activity, better selectivity and/or better preparability, are inventive compounds of the general formula (I) given or salts thereof or the inventive use thereof in which individual radicals have one of the preferred meanings already specified or specified below, or in particular those in which one or more of the preferred meanings already specified or specified below occur in combination.

With regard to the compounds of the invention, the terms used above and further down will be elucidated. These are familiar to the person skilled in the art and especially have the definitions elucidated hereinafter:

Unless defined differently, names of chemical groups are generally to be understood such that attachment to the skeleton or the remainder of the molecule is via the structural element of the relevant chemical group mentioned last, i.e. for example in the case of (C₁-C₄)-alkoxy via the oxygen atom and in the case of carboxy-(C₁-C₄)-alkyl or (C₁-C₄)-alkoxy-(C₁-C₄)-alkyl in each case via the carbon atom of the alkyl group.

According to the invention, “alkylsulfonyl”—on its own or as part of a chemical group - represents straight-chain or branched alkylsulfonyl, preferably having 1 to 4 carbon atoms, for example (but not limited to) (C₁-C₄)-alkylsulfonyl such as methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl.

According to the invention, “alkylthio”—on its own or as part of a chemical group - represents straight-chain or branched S-alkyl, preferably having 1 to 4 carbon atoms, such as (C₁-C₄)-alkylthio, for example (but not limited to) (C₁-C₄)-alkylthio such as methylthio, ethylthio, propylthio, 1-methylethylthio, butylthio, 1-methylpropylthio, 2-methylpropylthio, 1,1-dimethylethylthio.

According to the invention, “alkylsulfinyl (alkyl-S(═S)—)”, unless defined differently elsewhere, represents alkyl radicals bonded to the skeleton via —S(═S)—, such as (C₁-C₄)-alkylsulfinyl, for example (but not limited to) (C₁-C₄)-alkylsulfinyl such as methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl.

“Alkoxy” denotes an alkyl radical attached via an oxygen atom, for example (but not limited to) (C₁-C₄)-alkoxy such as methoxy, ethoxy, propoxy, 1-methylethoxy, butoxy, 1-methylpropoxy, 2-methylpropoxy, 1,1-dimethylethoxy.

According to the invention, “alkylcarbonyl” (alkyl-C(═O)—), unless defined differently elsewhere, represents alkyl radicals bonded to the skeleton via —C(═O)—, such as (C₁-C₄)-alkylcarbonyl. The number of the carbon atoms here relates to the alkyl radical in the alkylcarbonyl group.

“Alkoxycarbonyl (alkyl-O—C(═O)—)”, unless defined differently elsewhere: alkyl radicals bonded to the skeleton via —O—C(═O)—, such as (C₁-C₄)-alkoxycarbonyl. The number of the carbon atoms here relates to the alkyl radical in the alkoxycarbonyl group.

“Alkoxythiocarbonyl (Alkyl-O—C(═S)—)”, unless defined differently elsewhere: alkyl radicals bonded to the skeleton via —O—C(═S)—, such as (C₁-C₄)-alkoxythiocarbonyl. The number of the carbon atoms here relates to the alkyl radical in the alkoxythiocarbonyl group.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” denotes a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partially or fully substituted by identical or different halogen atoms, for example monohaloalkyl such as CH₂CH₂Cl, CH₂CH₂Br, CHC₁CH₃, CH₂C₁, CH₂F; dihaloalkyl such as CHF₂, CHCl₂; perhaloalkyl such as CF₃, CCl₃, CCIF₂, CBrF₂, CFCl₂, CF₂CClF₂, CF₂CClFCF₃; polyhaloalkyl such as CH₂CHFC₁, CF₂CClFH, CF2CBrFH, CH₂CF3; the term perhaloalkyl also encompasses the term perfluoroalkyl.

“Haloalkoxy” is, for example, OCF₃, OCHF₂, OCH₂F, OCF₂CF₃, OCH₂CF₃ and OCH₂CH₂Cl; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl) and 1,2-butadienyl. Alkenyl denotes, for example, vinyl, which can optionally be substituted by further alkyl radicals, for example (but not limited to) (C₂-C₄)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl. (C₂-C₄)-Alkynyl denotes, for example, ethynyl, 1-propynyl, 2-propynyl, 1-butynyl, 2-butynyl, 3-butynyl, 1-methyl-2-propynyl.

The term “cycloalkyl” refers to a carbocyclic saturated ring system having preferably 3-6 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-l-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl and bicyclo[2.1.1]hexyl, but also systems such as 1,1′-bi(cyclopropyl)-1-yl, 1,1′-bi(cyclopropyl)-2-yl. The term “(C₃-C₆)-cycloalkyl” is a brief notation for cycloalkyl having three to 6 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

According to the invention, “haloalkylthio”—on its own or as part of a chemical group—represents straight-chain or branched S-haloalkyl, preferably having 1 to 4 carbon atoms, such as (C₁-C₄)-for example (but not limited to) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” denotes cycloalkyl partly or fully substituted by identical or different halogen atoms, such as F, Cl and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-l-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

According to the invention, “trialkylsilyl”—on its own or as part of a chemical group—represents straight-chain or branched Si-alkyl, preferably having 1 to 6 carbon atoms, such as tri-[C₁-C₂)-alkyl]silyl, for example (but not limited to) trimethylsilyl, triethylsilyl.

If a collective term for a substituent, for example (C₁-C₄)-alkyl, is at the end of a composite substituent, such as for example in (C₃-C₆)-cycloalkyl-(C₁-C₄)-alkyl, the constituent at the start of the composite substituent, for example the (C₃-C₆)-cycloalkyl, may be mono- or polysubstituted identically or differently and independently by the latter substituent, in the present example (C₁-C₄)-alkyl.

Unless defined differently, the definition for collective terms also applies to these collective terms in composite substituents. Example: The definition of (C₁-C₄)-alkyl also applies to (C₁-C₄)-alkyl as component of a composite substituent such as, for example, (C₃-C₆)-cycloalkyl-(C₁-C₄)-alkyl.

If the compounds can form, through a hydrogen shift, tautomers whose structure would not formally be covered by the general formula (I), these tautomers are nevertheless encompassed by the definition of the inventive compounds of the general formula (I), unless a particular tautomer is under consideration. For example, many carbonyl compounds may be present both in the keto form and in the enol form, both forms being encompassed by the definition of the compound of the general formula (I).

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

If the compounds are obtained as solids, the purification can also be carried out by recrystallization or digestion. If individual compounds (I) cannot be obtained in a satisfactory manner by the routes described below, they can be prepared by derivatization of other compounds (I).

Suitable isolation methods, purification methods and methods for separating stereoisomers of compounds of the general formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

The present invention also claims processes for preparing the inventive compounds of the general formula (I).

The inventive compounds of the general formula (I) can be prepared, inter alia, using known processes. The synthesis routes used and examined proceed from commercially available or easily preparable building blocks. In the schemes which follow, the moieties R¹, R², R³, R⁴ and n of the general formula (I) have the meanings defined above, unless illustrative but non-limiting definitions are given.

Compounds according to the invention can be prepared, for example, by the method specified in scheme 1.

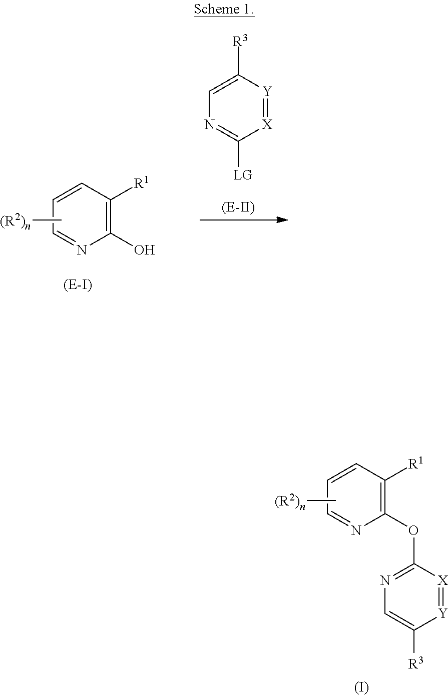

The heteroaryloxypyridines of the general formula (I) can be prepared via an alkylation of the hydroxypyridines (E-I) in the presence of bases with the pyridine, pyrimidine or pyrazine (E-II), where LG is a leaving group. The base may be a carbonate salt of an alkali metal (for example sodium, potassium or cesium), or silver carbonate. The reactions are generally carried out in an organic solvent, for example acetonitrile, butyronitrile, dimethylformamide or 1-methyl-2-pyrrolidone, at temperatures between 0° C. and the boiling point of the solvent. The R¹, R², R³, X and Y radicals and the index n mentioned in scheme 1 conform to the definitions given above.

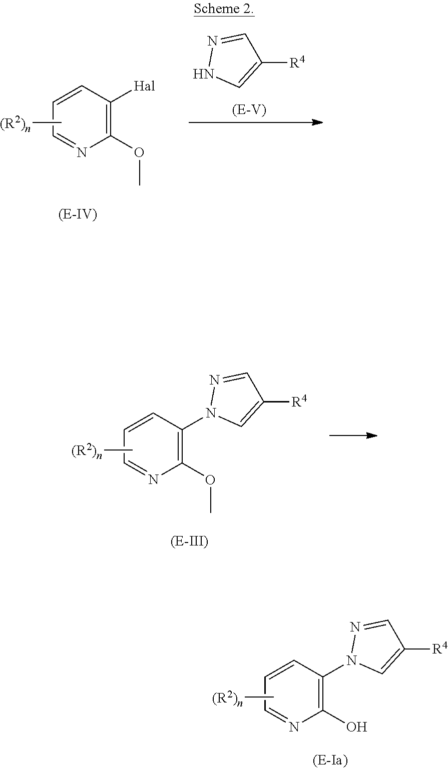

The pyridines of the general formula (E-Ia) can be prepared by demethylation of the pyridines (E-III) in the presence of either hydrobromic acid and acetic acid or boron tribromide (scheme 2). The reactions with boron tribromide are generally carried out in an organic solvent, for example dichloromethane, at temperatures between 0° C. and the boiling point of the solvent.

The pyridines of the general formula (E-III) can be prepared by a coupling of the pyridines (E-IV) with the pyrazole (E-V) in the presence of catalysts, ligands and bases, where Hal is a halogen such as iodine or bromine. The catalyst may be a copper salt (for example Cul). The ligand may be an amine (for example N,N ′-dimethylethane-1,2-diamine or N,N-dimethylglycine). The base may be a carbonate salt of an alkali metal (for example sodium, potassium or cesium). The reactions are generally conducted in an organic solvent, for example 1,4-dioxane, dimethylformamide, or dimethylacetamide, at temperatures between 0° C. and the boiling point of the solvent.

The synthesis of the pyridines (E-IV) is known to those skilled in the art and is described, for example, in WO2011/48525 and US2013/143907. The R² and R⁴ radicals and the index n mentioned in scheme 2 conform to the definitions given above.

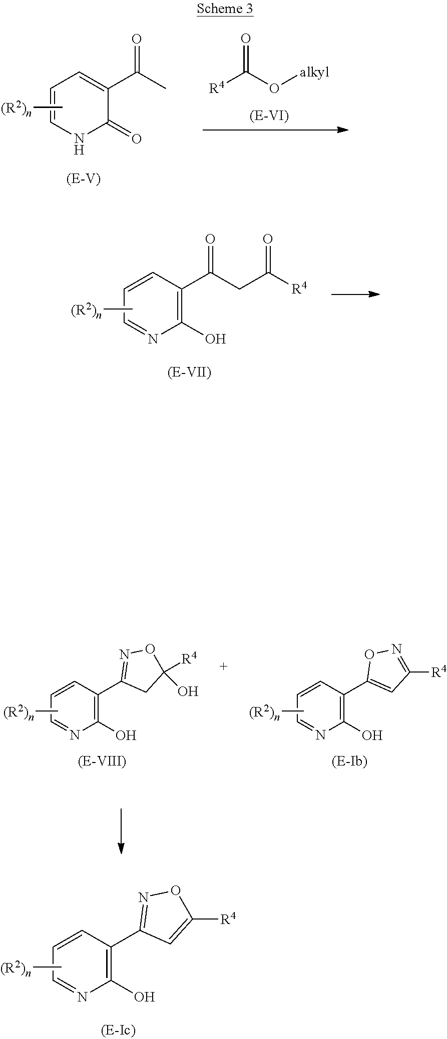

The pyridines of the general formula (E-Ic) can be prepared via a dehydrogenation of the pyridines (E-VIII) under acidic or basic conditions. The reactions under acidic conditions are performed in the presence of acids (for example hydrochloric acid and acetic acid). The reactions under basic conditions are performed in the presence of bases (for example a carbonate salt of an alkali metal) and an organic solvent (for example acetonitrile or dimethylformamide), at temperatures between 0° C. and the boiling point of the solvent.

The pyridines of the general formula (E-Ib) and (E-VIII) can be prepared via a reaction of the diketones (E-VII) with hydroxylamine or hydroxylamine hydrochloride. Reactions are generally carried out in an organic solvent, for example methanol or ethanol, at temperatures between 0° C. and the boiling point of the solvent.

The diketones of the general formula (E-VII) can be prepared via a reaction of the ketones (E-V) with esters (E-VI), in the presence of bases. The base may be a sodium salt of an alcohol (for example methanol or ethanol). Reactions are generally carried out in an organic solvent, for example tetrahydrofuran, methanol or ethanol, at temperatures between 0° C. and the boiling point of the solvent.

The synthesis of the ketones (E-V) is known to the person skilled in the art and is described, for example, in J. Org. Chem. 1970, 35, 3596-3600 and Chem. Pharm. Bull. 1976, 24, 303-309. The R² and R⁴ radicals and the index n mentioned in scheme 3 conform to the definitions given above.

### SYNTHESIS EXAMPLES

### Synthesis Example No. 1-8:

### Synthesis Stage 1: (Z)-1,1-Difluoro-4-hydroxy-4-(2-hydroxy-4,6-dimethyl-3-pyridyl)but-3-en-2-one (Intermediate A-01)

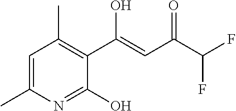 [SMILES: Cc1cc(C)c(/C(O)=C/C(=O)C(F)F)c(O)n1]

A mixture of 3-acetyl-4,6-dimethylpyridin-2(1H)-one (11.40 g, 69.0 mmol), ethyl 2,2-difluoroacetate (10.16 ml, 96.6 mmol) and sodium methoxide (30% solution in methanol, 37.3 g, 207.0 mmol) in 80 ml of tetrahydrofuran was heated at 60° C. for 4 h. The resulting reaction mixture was cooled down to room temperature, concentrated, diluted with water (25 ml) and then brought to pH 6-7 with 2M hydrochloric acid. Subsequent filtration resulted in isolation of (Z)-1,1-difluoro-4-hydroxy-4-(2-hydroxy-4,6-dimethyl-3-pyridyl)but-3-en-2-one. The yield was 7.10 g (42% of theory).

### Synthesis Stage 2: 4,6-Dimethyl-3-[3-(difluoromethyl)isoxazol-5-yl]pyridine-2-ol (Intermediate A-02) and 3-(2-hydroxy-4,6-dimethyl-3-pyridyl)-5-(difluoromethyl)-4H-isoxazole-5-ol (Intermediate A-03)

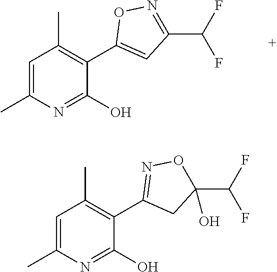 [SMILES: Cc1cc(C)c(-c2cc(C(F)F)no2)c(O)n1 ; Cc1cc(C)c(C2=NOC(O)(C(F)F)C2)c(O)n1]

A mixture of (Z)-1,1-difluoro-4-hydroxy-4-(2-hydroxy-4,6-dimethyl-3-pyridyl)but-3-en-2-one (Intermediate A-01, 1.50 g, 6.17 mmol) and hydroxylamine hydrochloride (860 mg, 12.3 mmol) in 10 ml of ethanol was heated at 90° C. for 3 h. The resulting reaction mixture was cooled down to room temperature. Subsequent filtration resulted in isolation of 4,6-dimethyl-3-[3-(difluoromethypisoxazol-5-yl]pyridine-2-ol. The yield was 1.07 g (72% of theory).

The filtrate was concentrated. Final purification of the resulting crude product by column chromatography (ethyl acetate/heptane gradient) resulted in isolation of 3-(2-hydroxy-4,6-dimethyl-3-pyridyl)-5-(difluoromethyl)-4H-isoxazole-5-ol. The yield was 180 mg (11% of theory).

### Synthesis Stage 3: 5-[2-(5-Chloropyrimidin-2-ypoxy-4,6-dimethyl-3-pyridyl]-3-(difluoromethypisoxazole (Synthesis example No. I-1)

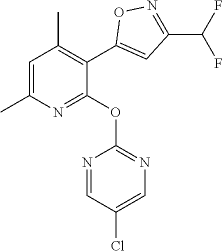 [SMILES: Cc1cc(C)c(-c2cc(C(F)F)no2)c(Oc2ncc(Cl)cn2)n1]

A mixture of 4,6-dimethyl-3[3-(difluoromethyflisoxazol-5-yl]pyridine-2-ol (Intermediate A-02, 150 mg, 0.59 mmol), 2-bromo-5-chloropyrimidine (120 mg, 0.52 mmol) and cesium carbonate (290 mg, 0.89 mmol) in 10 ml of dimethylacetamide was heated in a microwave at 100° C. for 60 minutes. The resulting reaction mixture was cooled down to room temperature, diluted with water and subsequently extracted repeatedly with dichloromethane. The combined organic phases were then washed with water, dried over magnesium sulfate, filtered and concentrated. Subsequent purification of the resulting crude product by column chromatography (ethyl acetate/heptane gradient) resulted in isolation of 5-[245-chloropyrimidin-2-yfloxy-4,6-dimethyl-3-pyridyl]-3-(difluoromethypisoxazole (synthesis example No. I-1). The yield was 64 mg (31% of theory).

### Synthesis Example No. I-15:

### Synthesis Stage 1: 3-15-(Difluoromethyl)isoxazol-3-yl1-4,6-dimethylpyridine-2-ol (Intermediate A-04)

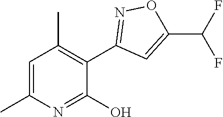 [SMILES: Cc1cc(C)c(-c2cc(C(F)F)on2)c(O)n1]

A mixture of 3-(2-hydroxy-4,6-dimethyl-3-pyridyl)-5-(difluoromethyl)-4H-isoxazole-5-ol (Intermediate A-03, 800 mg, 2.79 mmol), acetic acid (1.9 ml) and concentrated hydrochloric acid (0.7 ml) was heated at 80° C. for 24 h. The resulting reaction mixture was cooled to room temperature and diluted with 50 ml of water. Subsequent filtration resulted in isolation of 3-[5-(difluoromethyl)isoxazol-3-yl]-4,6-dimethylpyridine-2-ol. The yield was 690 mg (98% of theory).

### Synthesis stage 2: 5-(Difluoromethyl)-3-[2-(5-fluoropyrimidin-2-yfloxy-4,6-dimethyl-3-pyridyl]isoxazole (Synthesis example No. I-3)

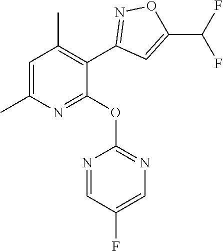 [SMILES: Cc1cc(C)c(-c2cc(C(F)F)on2)c(Oc2ncc(F)cn2)n1]

A mixture of 3-[5-(difluoromethyl)isoxazol-3-yl]-4,6-dimethylpyridine-2-ol (Intermediate A-04, 690 mg, 2.73 mmol), 2-chloro-5-fluoropyrimidine (435 mg, 3.28 mmol) and cesium carbonate (1.78 g, 5.46 mmol) in 8 ml of dimethylformamide was heated at 80° C. for 5 h. The resulting reaction mixture was cooled down to room temperature, diluted with water and subsequently extracted repeatedly with dichloromethane. The combined organic phases were then washed with water, dried over magnesium sulfate, filtered and concentrated. Final purification of the resulting crude product by column chromatography (ethyl acetate/heptane gradient) resulted in isolation of 5-(difluoromethyl)-342-(5-fluoropyrimidin-2-yfloxy-4,6-dimethyl-3-pyridyllisoxazole (Synthesis example No. I-3). The yield was 67 mg (7% of theory).

In analogy to the preparation examples cited above and recited at the appropriate point, the compounds of the general formula (I) specified hereinafter and shown in Table 1 are obtained.

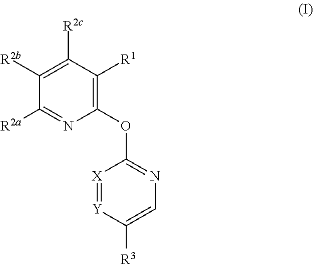

TABLE 1

| Example | | | | | | | |
|---|---|---|---|---|---|---|---|
| number | R¹ | R^{2a} | R^{2b} | R^{2c} | R³ | X | Y |
| I-1 | 3-CHF₂-5-isoxazolyl | Me | H | Me | Cl | N | CH |
| I-2 | 3-CF₃-5-isoxazolyl | Me | H | Me | Cl | N | CH |
| I-3 | 5-CHF₂-3-isoxazolyl | Me | H | Me | F | N | CH |
| I-4 | 3-CHF₂-5-isoxazolyl | Me | H | Me | F | N | CH |
| I-5 | 3-CHFCl-5-isoxazolyl | Me | H | Me | Cl | N | CH |
| I-6 | 5-CF₃-3-isoxazolyl | Me | H | Me | F | N | CH |
| I-7 | 5-CF₃-3-isoxazolyl | Me | H | Me | Cl | N | CH |
| I-8 | 3-CF₂Cl-5-isoxazolyl | Me | H | Me | Cl | N | CH |
| I-9 | 3-CHF₂-5-isoxazolyl | Me | H | H | F | N | CH |
| I-10 | 5-CHF₂-3-isoxazolyl | Me | H | Me | Cl | N | CH |
| I-11 | 3-CHF₂-5-isoxazoly1 | Me | H | Me | Cl | CH | N |
| I-12 | 5-CHF₂-3-isoxazolyl | Me | H | H | Cl | N | CH |
| I-13 | 5-CHF₂-3-isoxazolyl | Me | H | H | F | N | CH |
| I-14 | 3-CHF₂-5-isoxazolyl | Me | H | Me | Cl | CF | CH |
| I-15 | 5-CF₂Cl-3-isoxazolyl | Me | H | Me | F | N | CH |
| I-16 | 3-CF₃-5-isoxazolyl | Me | H | Me | F | N | CH |
| I-17 | 3-CF₃-5-isoxazolyl | Me | H | Me | Br | N | CH |
| I-18 | 3-CF₃-5-isoxazolyl | Me | H | Me | Cl | CH | N |
| I-19 | 3-CF₃-5-isoxazolyl | Me | H | Me | Cl | CF | CH |
| I-20 | 3-CF₃-5-isoxazolyl | Me | H | Me | Cl | CH | CH |
| I-21 | 3-CF₃-5-isoxazolyl | H | H | Me | F | N | CH |
| I-22 | 3-CF₃-5-isoxazolyl | H | H | Me | Cl | N | CH |
| I-23 | 3-CF₃-5-isoxazolyl | Me | H | Cl | F | N | CH |
| I-24 | 3-CF₃-5-isoxazolyl | Me | H | Cl | Cl | N | CH |
| I-25 | 3-CF₃-5-isoxazoly1 | Me | H | Br | F | N | CH |
| I-26 | 3-CF₃-5-isoxazolyl | Me | H | Br | Cl | N | CH |
| I-27 | 3-CF₃-5-isoxazolyl | Me | H | CN | F | N | CH |
| I-28 | 3-CF₃-5-isoxazolyl | Me | H | CN | Cl | N | CH |
| I-29 | 5-CF₃-3-isoxazolyl | Me | H | Me | Br | N | CH |
| I-30 | 5-CF₃-3-isoxazolyl | Me | H | Me | Cl | CH | N |
| I-31 | 5-CF₃-3-isoxazolyl | Me | H | Me | Cl | CF | CH |
| I-32 | 5-CF₃-3-isoxazolyl | Me | H | Me | Cl | CH | CH |
| I-33 | 5-CF₃-3-isoxazolyl | H | H | Me | F | N | CH |
| I-34 | 5-CF₃-3-isoxazolyl | H | H | Me | Cl | N | CH |
| I-35 | 5-CF₃-3-isoxazolyl | Me | H | Cl | F | N | CH |
| I-36 | 5-CF₃-3-isoxazolyl | Me | H | Cl | Cl | N | CH |
| I-37 | 5-CF₃-3-isoxazolyl | Me | H | Br | F | N | CH |
| I-38 | 5-CF₃-3-isoxazolyl | Me | H | Br | Cl | N | CH |
| I-39 | 5-CF₃-3-isoxazolyl | Me | H | CN | F | N | CH |
| I-40 | 5-CF₃-3-isoxazolyl | Me | H | CN | Cl | N | CH |
| I-41 | 3-CHF₂-5-isoxazolyl | Me | H | Me | Br | N | CH |
| I-42 | 3-CHF₂-5-isoxazolyl | Me | H | Me | Cl | CH | N |
| I-43 | 3-CHF₂-5-isoxazolyl | Me | H | Me | Cl | CH | CH |
| I-44 | 3-CHF₂-5-isoxazolyl | Me | H | Me | NO₂ | N | CH |
| I-45 | 3-CHF₂-5-isoxazolyl | Me | H | Me | CF₃ | N | CH |
| I-46 | 3-CHF₂-5-isoxazolyl | Me | H | Me | CN | N | CH |
| I-47 | 3-CHF₂-5-isoxazolyl | H | H | Me | F | N | CH |
| I-48 | 3-CHF₂-5-isoxazolyl | H | H | Me | Cl | N | CH |
| I-49 | 3-CHF₂-5-isoxazolyl | Me | H | Cl | F | N | CH |
| I-50 | 3-CHF₂-5-isoxazolyl | Me | H | Cl | Cl | N | CH |
| I-51 | 3-CHF₂-5-isoxazolyl | Me | H | Br | F | N | CH |
| I-52 | 3-CHF₂-5-isoxazolyl | Me | H | Br | Cl | N | CH |
| I-53 | 3-CHF₂-5-isoxazolyl | Me | H | CN | F | N | CH |
| I-54 | 3-CHF₂-5-isoxazolyl | Me | H | CN | Cl | N | CH |
| I-55 | 5-CHF₂-3-isoxazolyl | Me | H | Me | Br | N | CH |
| I-56 | 5-CHF₂-3-isoxazolyl | Me | H | Me | Cl | CH | N |
| I-57 | 5-CHF₂-3-isoxazolyl | Me | H | Me | Cl | CF | CH |
| I-58 | 5-CHF₂-3-isoxazolyl | Me | H | Me | Cl | CH | CH |
| I-59 | 5-CHF₂-3-isoxazolyl | H | H | Me | F | N | CH |
| I-60 | 5-CHF₂-3-isoxazolyl | H | H | Me | Cl | N | CH |
| I-61 | 5-CHF₂-3-isoxazolyl | Me | H | Cl | F | N | CH |
| I-62 | 5-CHF₂-3-isoxazolyl | Me | H | Cl | Cl | N | CH |
| I-63 | 5-CHF₂-3-isoxazolyl | Me | H | Br | F | N | CH |
| I-64 | 5-CHF₂-3-isoxazolyl | Me | H | Br | Cl | N | CH |
| I-65 | 5-CHF₂-3-isoxazolyl | Me | H | CN | F | N | CH |
| I-66 | 5-CHF₂-3-isoxazolyl | Me | H | CN | Cl | N | CH |
| I-67 | 3-CHFCl-5-isoxazolyl | Me | H | Me | F | N | CH |
| I-68 | 3-CHFCl-5-isoxazolyl | Me | H | Me | Br | N | CH |
| I-69 | 3-CHFCl-5-isoxazolyl | H | H | Me | F | N | CH |
| I-70 | 3-CHFCl-5-isoxazolyl | H | H | Me | Cl | N | CH |
| I-71 | 3-CHFCl-5-isoxazolyl | Me | H | Cl | F | N | CH |
| I-72 | 3-CHFCl-5-isoxazolyl | Me | H | Cl | Cl | N | CH |
| I-73 | 3-CHFCl-5-isoxazolyl | Me | H | Br | F | N | CH |
| I-74 | 3-CHFCl-5-isoxazolyl | Me | H | Br | Cl | N | CH |
| I-75 | 3-CHFCl-5-isoxazolyl | Me | H | CN | F | N | CH |
| I-76 | 3-CHFCl-5-isoxazolyl | Me | H | CN | Cl | N | CH |
| I-77 | 5-CHFCl-3-isoxazolyl | Me | H | Me | F | N | CH |
| I-78 | 5-CHFCl-3-isoxazolyl | Me | H | Me | Cl | N | CH |
| I-79 | 5-CHFCl-3-isoxazolyl | Me | H | Me | Cl | CF | CH |
| I-80 | 5-CHFCl-3-isoxazolyl | H | H | Me | F | N | CH |
| I-81 | 5-CHFCl-3-isoxazolyl | H | H | Me | Cl | N | CH |
| I-82 | 5-CHFCl-3-isoxazolyl | Me | H | Cl | F | N | CH |
| I-83 | 5-CHFCl-3-isoxazolyl | Me | H | Cl | Cl | N | CH |
| I-84 | 5-CHFCl-3-isoxazolyl | Me | H | Br | F | N | CH |
| I-85 | 5-CHFCl-3-isoxazolyl | Me | H | Br | Cl | N | CH |
| I-86 | 5-CHFCl-3-isoxazolyl | Me | H | CN | F | N | CH |
| I-87 | 5-CHFCl-3-isoxazolyl | Me | H | CN | Cl | N | CH |
| I-88 | 3-CF₂Cl-5-isoxazolyl | Me | H | Me | F | N | CH |
| I-89 | 3-CF₂Cl-5-isoxazolyl | Me | H | Me | Br | N | CH |
| I-90 | 3-CF₂Cl-5-isoxazolyl | Me | H | Me | Cl | CF | CH |
| I-91 | 3-CF₂Cl-5-isoxazolyl | Me | H | Me | Cl | CH | CH |
| I-92 | 3-CF₂Cl-5-isoxazolyl | Me | H | Me | Cl | CH | N |
| I-93 | 3-CF₂Cl-5-isoxazolyl | H | H | Me | F | N | CH |
| I-94 | 3-CF₂Cl-5-isoxazolyl | H | H | Me | Cl | N | CH |
| I-95 | 3-CF₂Cl-5-isoxazolyl | Me | H | Cl | F | N | CH |
| I-96 | 3-CF₂Cl-5-isoxazolyl | Me | H | Cl | Cl | N | CH |
| I-97 | 3-CF₂Cl-5-isoxazolyl | Me | H | Br | F | N | CH |
| I-98 | 3-CF₂Cl-5-isoxazolyl | Me | H | Br | Cl | N | CH |
| I-99 | 3-CF₂Cl-5-isoxazolyl | Me | H | CN | F | N | CH |
| I-100 | 3-CF₂Cl-5-isoxazolyl | Me | H | CN | Cl | N | CH |
| I-101 | 5-CF₂Cl-3-isoxazolyl | Me | H | Me | Cl | N | CH |
| I-102 | 5-CF₂Cl-3-isoxazolyl | Me | H | Me | Br | N | CH |
| I-103 | 5-CF₂Cl-3-isoxazolyl | Me | H | Me | Cl | CF | CH |
| I-104 | 5-CF₂Cl-3-isoxazolyl | H | H | Me | F | N | CH |
| I-105 | 5-CF₂Cl-3-isoxazolyl | H | H | Me | Cl | N | CH |
| I-106 | 5-CF₂Cl-3-isoxazolyl | Me | H | Cl | F | N | CH |
| I-107 | 5-CF₂Cl-3-isoxazolyl | Me | H | Cl | Cl | N | CH |
| I-108 | 5-CF₂Cl-3-isoxazolyl | Me | H | Br | F | N | CH |
| I-109 | 5-CF₂Cl-3-isoxazolyl | Me | H | Br | Cl | N | CH |
| I-110 | 5-CF₂Cl-3-isoxazolyl | Me | H | CN | F | N | CH |
| I-111 | 5-CF₂Cl-3-isoxazolyl | Me | H | CN | Cl | N | CH |
| I-112 | 3-CCl₃-5-isoxazolyl | Me | H | Me | F | N | CH |
| I-113 | 3-CCl₃-5-isoxazolyl | Me | H | Me | Cl | N | CH |
| I-114 | 3-CCl₃-5-isoxazolyl | Me | H | Me | Cl | CF | CH |
| I-115 | 3-CCl₃-5-isoxazolyl | H | H | Me | F | N | CH |
| I-116 | 3-CCl₃-5-isoxazolyl | H | H | Me | Cl | N | CH |
| I-117 | 3-CCl₃-5-isoxazolyl | Me | H | Cl | F | N | CH |
| I-118 | 3-CCl₃-5-isoxazolyl | Me | H | Cl | Cl | N | CH |
| I-119 | 3-CCl₃-5-isoxazolyl | Me | H | Br | F | N | CH |
| I-120 | 3-CCl₃-5-isoxazolyl | Me | H | Br | Cl | N | CH |
| I-121 | 3-CCl₃-5-isoxazolyl | Me | H | CN | F | N | CH |
| I-122 | 3-CCl₃-5-isoxazolyl | Me | H | CN | Cl | N | CH |
| I-123 | 5-CCl₃-3-isoxazolyl | Me | H | Me | F | N | CH |
| I-124 | 5-CCl₃-3-isoxazolyl | Me | H | Me | Cl | N | CH |
| I-125 | 5-CCl₃-3-isoxazolyl | Me | H | Me | Cl | CF | CH |
| I-126 | 5-CCl₃-3-isoxazolyl | H | H | Me | F | N | CH |
| I-127 | 5-CCl₃-3-isoxazolyl | H | H | Me | Cl | N | CH |
| I-128 | 5-CCl₃-3-isoxazolyl | Me | H | Cl | F | N | CH |
| I-129 | 5-CCl₃-3-isoxazolyl | Me | H | Cl | Cl | N | CH |
| I-130 | 5-CCl₃-3-isoxazolyl | Me | H | Br | F | N | CH |
| I-131 | 5-CCl₃-3-isoxazolyl | Me | H | Br | Cl | N | CH |
| I-132 | 5-CCl₃-3-isoxazolyl | Me | H | CN | F | N | CH |
| I-133 | 5-CCl₃-3-isoxazolyl | Me | H | CN | Cl | N | CH |
| I-134 | 3-CHCl₂-5-isoxazolyl | Me | H | Me | F | N | CH |
| I-135 | 3-CHCl₂-5-isoxazolyl | Me | H | Me | Cl | N | CH |
| I-136 | 3-CHCl₂-5-isoxazolyl | Me | H | Me | Cl | CF | CH |
| I-137 | 3-CHCl₂-5-isoxazolyl | H | H | Me | F | N | CH |
| I-138 | 3-CHCl₂-5-isoxazolyl | H | H | Me | Cl | N | CH |
| I-139 | 3-CHCl₂-5-isoxazolyl | Me | H | Cl | F | N | CH |
| I-140 | 3-CHCl₂-5-isoxazolyl | Me | H | Cl | Cl | N | CH |
| I-141 | 3-CHCl₂-5-isoxazolyl | Me | H | Br | F | N | CH |
| I-142 | 3-CHCl₂-5-isoxazolyl | Me | H | Br | Cl | N | CH |
| I-143 | 3-CHCl₂-5-isoxazolyl | Me | H | CN | F | N | CH |
| I-144 | 3-CHCl₂-5-isoxazolyl | Me | H | CN | Cl | N | CH |
| I-145 | 5-CHCl₂-3-isoxazolyl | Me | H | Me | F | N | CH |
| I-146 | 5-CHCl₂-3-isoxazoly1 | Me | H | Me | Cl | N | CH |
| I-147 | 5-CHCl₂-3-isoxazolyl | Me | H | Me | Cl | CF | CH |
| I-148 | 5-CHCl₂-3-isoxazolyl | H | H | Me | F | N | CH |
| I-149 | 5-CHCl₂-3-isoxazolyl | H | H | Me | Cl | N | CH |
| I-150 | 5-CHCl₂-3-isoxazolyl | Me | H | Cl | F | N | CH |
| I-151 | 5-CHCl₂-3-isoxazolyl | Me | H | Cl | Cl | N | CH |
| I-152 | 5-CHCl₂-3-isoxazolyl | Me | H | Br | F | N | CH |
| I-153 | 5-CHCl₂-3-isoxazolyl | Me | H | Br | Cl | N | CH |
| I-154 | 5-CHCl₂-3-isoxazolyl | Me | H | CN | F | N | CH |
| I-155 | 5-CHCl₂-3-isoxazolyl | Me | H | CN | Cl | N | CH |
| I-156 | 3-CF₂Br-5-isoxazolyl | Me | H | Me | F | N | CH |
| I-157 | 3-CF₂Br-5-isoxazolyl | Me | H | Me | Cl | N | CH |
| I-158 | 3-CF₂Br-5-isoxazolyl | Me | H | Me | Cl | CF | CH |
| I-159 | 3-CF₂Br-5-isoxazolyl | H | H | Me | F | N | CH |
| I-160 | 3-CF₂Br-5-isoxazolyl | H | H | Me | Cl | N | CH |
| I-161 | 3-CF₂Br-5-isoxazolyl | Me | H | Cl | F | N | CH |
| I-162 | 3-CF₂Br-5-isoxazolyl | Me | H | Cl | Cl | N | CH |
| I-163 | 3-CF₂Br-5-isoxazolyl | Me | H | Br | F | N | CH |
| I-164 | 3-CF₂Br-5-isoxazolyl | Me | H | Br | Cl | N | CH |
| I-165 | 3-CF₂Br-5-isoxazolyl | Me | H | CN | F | N | CH |
| I-166 | 3-CF₂Br-5-isoxazolyl | Me | H | CN | Cl | N | CH |
| I-167 | 5-CF₂Br-3-isoxazolyl | Me | H | Me | F | N | CH |
| I-168 | 5-CF₂Br-3-isoxazolyl | Me | H | Me | Cl | N | CH |
| I-169 | 5-CF₂Br-3-isoxazolyl | Me | H | Me | Cl | CF | CH |
| I-170 | 5-CF₂Br-3-isoxazolyl | H | H | Me | F | N | CH |
| I-171 | 5-CF₂Br-3-isoxazolyl | H | H | Me | Cl | N | CH |
| I-172 | 5-CF₂Br-3-isoxazolyl | Me | H | Cl | F | N | CH |
| I-173 | 5-CF₂Br-3-isoxazolyl | Me | H | Cl | Cl | N | CH |
| I-174 | 5-CF₂Br-3-isoxazolyl | Me | H | Br | F | N | CH |
| I-175 | 5-CF₂Br-3-isoxazolyl | Me | H | Br | Cl | N | CH |
| I-176 | 5-CF₂Br-3-isoxazolyl | Me | H | CN | F | N | CH |
| I-177 | 5-CF₂Br-3-isoxazolyl | Me | H | CN | Cl | N | CH |
| I-178 | 3-cPr-5-isoxazolyl | Me | H | Me | F | N | CH |
| I-179 | 3-cPr-5-isoxazolyl | Me | H | Me | Cl | N | CH |
| I-180 | 3-cPr-5-isoxazolyl | Me | H | Me | Cl | CF | CH |
| I-181 | 3-cPr-5-isoxazolyl | H | H | Me | F | N | CH |
| I-182 | 3-cPr-5-isoxazolyl | H | H | Me | Cl | N | CH |
| I-183 | 3-cPr-5-isoxazolyl | Me | H | Cl | F | N | CH |
| I-184 | 3-cPr-5-isoxazolyl | Me | H | Cl | Cl | N | CH |
| I-185 | 3-cPr-5-isoxazolyl | Me | H | Br | F | N | CH |
| I-186 | 3-cPr-5-isoxazolyl | Me | H | Br | Cl | N | CH |
| I-187 | 3-cPr-5-isoxazolyl | Me | H | CN | F | N | CH |
| I-188 | 3-cPr-5-isoxazolyl | Me | H | CN | Cl | N | CH |
| I-189 | 5-cPr-3-isoxazolyl | Me | H | Me | F | N | CH |
| I-190 | 5-cPr-3-isoxazolyl | Me | H | Me | Cl | N | CH |
| I-191 | 5-cPr-3-isoxazolyl | Me | H | Me | Cl | CF | CH |
| I-192 | 5-cPr-3-isoxazolyl | H | H | Me | F | N | CH |
| I-193 | 5-cPr-3-isoxazolyl | H | H | Me | Cl | N | CH |
| I-194 | 5-cPr-3-isoxazolyl | Me | H | Cl | F | N | CH |
| I-195 | 5-cPr-3-isoxazolyl | Me | H | Cl | Cl | N | CH |
| I-196 | 5-cPr-3-isoxazolyl | Me | H | Br | F | N | CH |
| I-197 | 5-cPr-3-isoxazolyl | Me | H | Br | Cl | N | CH |
| I-198 | 5-cPr-3-isoxazolyl | Me | H | CN | F | N | CH |
| I-199 | 5-cPr-3-isoxazolyl | Me | H | CN | Cl | N | CH |
| I-200 | 3-CH₂cPr-5-isoxazolyl | Me | H | Me | F | N | CH |
| I-201 | 3-CH₂cPr-5-isoxazolyl | Me | H | Me | Cl | N | CH |
| I-202 | 3-CH₂cPr-5-isoxazolyl | Me | H | Me | Cl | CF | CH |
| I-203 | 3-CH₂cPr-5-isoxazolyl | H | H | Me | F | N | CH |
| I-204 | 3-CH₂cPr-5-isoxazolyl | H | H | Me | Cl | N | CH |
| I-205 | 3-CH₂cPr-5-isoxazolyl | Me | H | Cl | F | N | CH |
| I-206 | 3-CH₂cPr-5-isoxazolyl | Me | H | Cl | Cl | N | CH |
| I-207 | 3-CH₂cPr-5-isoxazolyl | Me | H | Br | F | N | CH |
| I-208 | 3-CH₂cPr-5-isoxazolyl | Me | H | Br | Cl | N | CH |
| I-209 | 3-CH₂cPr-5-isoxazolyl | Me | H | CN | F | N | CH |
| I-210 | 3-CH₂cPr-5-isoxazolyl | Me | H | CN | Cl | N | CH |
| I-211 | 5-CH₂cPr-3-isoxazolyl | Me | H | Me | F | N | CH |
| I-212 | 5-CH₂cPr-3-isoxazolyl | Me | H | Me | Cl | N | CH |
| I-213 | 5-CH₂cPr-3-isoxazolyl | Me | H | Me | Cl | CF | CH |
| I-214 | 5-CH₂cPr-3-isoxazolyl | H | H | Me | F | N | CH |
| I-215 | 5-CH₂cPr-3-isoxazolyl | H | H | Me | Cl | N | CH |
| I-216 | 5-CH₂cPr-3-isoxazolyl | Me | H | Cl | F | N | CH |
| I-217 | 5-CH₂cPr-3-isoxazolyl | Me | H | Cl | Cl | N | CH |
| I-218 | 5-CH₂cPr-3-isoxazolyl | Me | H | Br | F | N | CH |
| I-219 | 5-CH₂cPr-3-isoxazolyl | Me | H | Br | Cl | N | CH |
| I-220 | 5-CH₂cPr-3-isoxazolyl | Me | H | CN | F | N | CH |
| I-221 | 5-CH₂cPr-3-isoxazolyl | Me | H | CN | Cl | N | CH |
| I-222 | 3-CH₂OMe-5-isoxazolyl | Me | H | Me | F | N | CH |
| I-223 | 3-CH₂OMe-5-isoxazolyl | Me | H | Me | Cl | N | CH |
| I-224 | 3-CH₂OMe-5-isoxazolyl | Me | H | Me | Cl | CF | CH |
| I-225 | 3-CH₂OMe-5-isoxazolyl | H | H | Me | F | N | CH |
| I-226 | 3-CH₂OMe-5-isoxazolyl | H | H | Me | Cl | N | CH |
| I-227 | 3-CH₂OMe-5-isoxazolyl | Me | H | Cl | F | N | CH |
| I-228 | 3-CH₂OMe-5-isoxazolyl | Me | H | Cl | Cl | N | CH |
| I-229 | 3-CH₂OMe-5-isoxazolyl | Me | H | Br | F | N | CH |
| I-230 | 3-CH₂OMe-5-isoxazolyl | Me | H | Br | Cl | N | CH |
| I-231 | 3-CH₂OMe-5-isoxazolyl | Me | H | CN | F | N | CH |
| I-232 | 3-CH₂OMe-5-isoxazolyl | Me | H | CN | Cl | N | CH |
| I-233 | 5-CH₂OMe-3-isoxazolyl | Me | H | Me | F | N | CH |
| I-234 | 5-CH₂OMe-3-isoxazolyl | Me | H | Me | Cl | N | CH |
| I-235 | 5-CH₂OMe-3-isoxazolyl | Me | H | Me | Cl | CF | CH |
| I-236 | 5-CH₂OMe-3-isoxazolyl | H | H | Me | F | N | CH |
| I-237 | 5-CH₂OMe-3-isoxazolyl | H | H | Me | Cl | N | CH |
| I-238 | 5-CH₂OMe-3-isoxazolyl | Me | H | Cl | F | N | CH |
| I-239 | 5-CH₂OMe-3-isoxazolyl | Me | H | Cl | Cl | N | CH |
| I-240 | 5-CH₂OMe-3-isoxazolyl | Me | H | Br | F | N | CH |
| I-241 | 5-CH₂OMe-3-isoxazolyl | Me | H | Br | Cl | N | CH |
| I-242 | 5-CH₂OMe-3-isoxazolyl | Me | H | CN | F | N | CH |
| I-243 | 5-CH₂OMe-3-isoxazolyl | Me | H | CN | Cl | N | CH |
| I-244 | 1-pyrazolyl | Me | H | Me | F | N | CH |
| I-245 | 1-pyrazolyl | Me | H | Me | Cl | N | CH |
| I-246 | 1-pyrazolyl | Me | H | Me | Cl | CF | CH |
| I-247 | 1-pyrazolyl | H | H | Me | F | N | CH |
| I-248 | 1-pyrazolyl | H | H | Me | Cl | N | CH |
| I-249 | 1-pyrazolyl | Me | H | Cl | F | N | CH |
| I-250 | 1-pyrazolyl | Me | H | Cl | Cl | N | CH |
| I-251 | 1-pyrazolyl | Me | H | Br | F | N | CH |
| I-252 | 1-pyrazolyl | Me | H | Br | Cl | N | CH |
| I-253 | 1-pyrazolyl | Me | H | CN | F | N | CH |
| I-254 | 1-pyrazolyl | Me | H | CN | Cl | N | CH |
| I-255 | 4-CF₃-1-pyrazolyl | Me | H | Me | F | N | CH |
| I-256 | 4-CF₃-1-pyrazolyl | Me | H | Me | Cl | N | CH |
| I-257 | 4-CF₃-1-pyrazolyl | Me | H | Me | Cl | CF | CH |
| I-258 | 4-CF₃-1-pyrazolyl | H | H | Me | F | N | CH |
| I-259 | 4-CF₃-1-pyrazolyl | H | H | Me | Cl | N | CH |
| I-260 | 4-CF₃-1-pyrazolyl | Me | H | Cl | F | N | CH |
| I-261 | 4-CF₃-1-pyrazolyl | Me | H | Cl | Cl | N | CH |
| I-262 | 4-CF₃-1-pyrazolyl | Me | H | Br | F | N | CH |
| I-263 | 4-CF₃-1-pyrazolyl | Me | H | Br | Cl | N | CH |
| I-264 | 4-CF₃-1-pyrazolyl | Me | H | CN | F | N | CH |
| I-265 | 4-CF₃-1-pyrazolyl | Me | H | CN | Cl | N | CH |
| I-266 | 4-Cl-1-pyrazolyl | Me | H | Me | F | N | CH |
| I-267 | 4-Cl-1-pyrazolyl | Me | H | Me | Cl | N | CH |
| I-268 | 4-Cl-1-pyrazolyl | Me | H | Me | Cl | CF | CH |
| I-269 | 4-Cl-1-pyrazolyl | H | H | Me | F | N | CH |
| I-270 | 4-Cl-1-pyrazolyl | H | H | Me | Cl | N | CH |
| I-271 | 4-Cl-1-pyrazolyl | Me | H | Cl | F | N | CH |
| I-272 | 4-Cl-1-pyrazolyl | Me | H | Cl | Cl | N | CH |
| I-273 | 4-Cl-1-pyrazolyl | Me | H | Br | F | N | CH |
| I-274 | 4-Cl-1-pyrazolyl | Me | H | Br | Cl | N | CH |
| I-275 | 4-Cl-1-pyrazolyl | Me | H | CN | F | N | CH |
| I-276 | 4-Cl-1-pyrazolyl | Me | H | CN | Cl | N | CH |
| I-277 | 4-Br-1-pyrazolyl | Me | H | Me | F | N | CH |
| I-278 | 4-Br-1-pyrazolyl | Me | H | Me | Cl | N | CH |
| I-279 | 4-Br-1-pyrazolyl | Me | H | Me | Cl | CF | CH |
| I-280 | 4-Br-1-pyrazolyl | H | H | Me | F | N | CH |
| I-281 | 4-Br-1-pyrazolyl | H | H | Me | Cl | N | CH |
| I-282 | 4-Br-1-pyrazolyl | Me | H | Cl | F | N | CH |
| I-283 | 4-Br-1-pyrazolyl | Me | H | Cl | Cl | N | CH |
| I-284 | 4-Br-1-pyrazolyl | Me | H | Br | F | N | CH |
| I-285 | 4-Br-1-pyrazolyl | Me | H | Br | Cl | N | CH |
| I-286 | 4-Br-1-pyrazolyl | Me | H | CN | F | N | CH |
| I-287 | 4-Br-1-pyrazolyl | Me | H | CN | Cl | N | CH |
| I-288 | 3-CHF₂-5-isoxazolyl | Me | H | Me | Cl | CCl | CH |
| I-289 | 3-CHF₂-5-isoxazolyl | Me | H | Me | F | CCN | CH |
| I-290 | 3-CHF₂-5-isoxazolyl | Me | H | Me | Cl | CCN | CH |
| I-291 | 3-CHF₂-5-isoxazolyl | Me | H | Me | Br | CCN | CH |
| I-292 | 3-CHF₂-5-isoxazolyl | Me | H | Me | CN | CH | CH |
| I-293 | 3-CHF₂-5-isoxazoly1 | Me | H | Me | F | CH | CH |
| I-294 | 4-I-1-pyrazolyl | Me | H | Me | F | N | CH |
| I-295 | 4-I-1-pyrazolyl | Me | H | Me | Cl | N | CH |
| I-296 | 3-cPr-5-isoxazolyl | Me | H | Me | Br | N | CH |
| I-297 | 3-(1-methylcyclopropyl)-5-isoxazolyl | Me | H | Me | Cl | N | CH |
| I-298 | 3-(1-methylcyclopropyl)-5-isoxazolyl | Me | H | Me | Br | N | CH |
| I-299 | 3-(1-methylcyclopropyl)-5-isoxazolyl | Me | H | Me | F | N | CH |
| I-300 | 3-CH₂cPr-5-isoxazolyl | Me | H | Me | Br | N | CH |
| I-301 | 3-(1-methylcyclopropyl)-5-isoxazolyl | Me | H | Me | Cl | CF | CH |

### NMR Data of Selected Examples

Selected detailed synthesis examples for the inventive compounds of the general formulas (I) are adduced below. The ¹H NMR spectroscopic data given for the chemical examples described in the following sections (400 MHz for ¹H NMR, solvent CDCl₃ or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quin=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported.

### Example No. I-1:

¹H-1-NMR (400 MHz, CDCl₃ δ, ppm) 8.51 (s, 2H), 7.07 (s, 1H), 6.76 (t, J=55 Hz, 1H), 6.74 (s, 1H), 2.49 (s, 3H), 2.48 (s, 3H).

### Example No. I-2:

¹H-NMR (400 MHz, CDCl₃ δ, ppm) 8.53 (s, 2H), 7.07 (s, 1H), 6.80 (s, 1H), 2.50 (s, 3H), 2.48 (s, 3H).

### Example No. I-3:

¹H-NMR (400 MHz, CDCl₃ δ, ppm) 8.42 (s, 2H), 7.40 (s, 1H), 7.08 (s, 1H), 6.64 (t, J=55 Hz, 1H), 2.52 (s, 3H), 2.52 (s, 31-1).

### Example No. I-4:

¹H-1-NMR (400 MHz, CDCl₃ δ, ppm) 8.44 (s, 2H), 7.05 (s, 1H), 6.76 (s, 1H), 6.76 (t, J=55 Hz, 1H), 10 2.48 (s, 3H), 2.47 (s, 3H).

### Example No. I-5:

¹H-NMR (400 MHz, CDCl₃ δ, ppm) 8.51 (s, 2H), 7.13 (d, J=50 Hz, 1H), 7.07 (s, 1H), 6.78 (s, 1H), 2.49 (s, 3H), 2.47 (s, 3H).

### Example No. I-6:

¹H-1-NMR (400 MHz, CDCl₃ δ, ppm) 8.43 (s, 2H), 7.51 (s, 1H), 7.09 (s, 1H), 2.52 (s, 3H), 2.52 (s, 3H).

### Example No. I-7:

¹H-NMR (400 MHz, CDCl₃ δ, ppm) 8.50 (s, 2H), 7.51 (s, 1H), 7.11 (s, 1H), 2.54 (s, 3H), 2.53 (s, 3H).

### Example No. I-8:

¹H-NMR (400 MHz, CDCl₃ δ, ppm) 8.52 (s, 2H), 7.07 (s, 1H), 6.79 (s, 1H), 2.50 (s, 3H), 2.49 (s, 3H).

### Example No. I-10:

¹H-NMR (400 MHz, CDCl₃ δ, ppm) 8.49 (s, 2H), 7.39 (s, 1H), 7.10 (s, 1H), 6.63 (t, J=55Hz, 1H), 2.53 (s, 3H), 2.52 (s, 3H).

### Example No. I-11:

¹H-NMR (400 MHz, CDCl₃ δ, ppm) 8.29 (s, 1H), 8.26 (s, 1H), 7.00 (s, 1H), 6.81 (t, J=55 Hz, 1H), 6.77 (s, 1H), 2.47 (s, 3H), 2.42 (s, 3H).

### Example No. I-12: ¹H-NMR (400 MHz, CDCl₃ δ, ppm) 8.58 (s, 2H), 8.30 (d, 1H), 7.22 (d, 1H), 6.98 (s, 1H), 6.63 (t, J=50 Hz, 1H), 2.52 (s, 3H).

### Example No. I-13:

¹H-NMR (400 MHz, CDCl₃ δ, ppm) 8.52 (s, 2H), 8.30 (d, 1H), 7.20 (d, 1H), 7.01 (s, 1H), 6.78 (t, J=55 Hz, 1H), 2.50 (s, 3H).

### Example No. I-14:

¹H-NMR (400 MHz, CDCl₃ δ, ppm) 8.09 (d, 1H), 7.56 (dd, I H), 6.94 (s, 1H), 6.84 (s, 1H), 6.81 (t, J=55 Hz, 1H), 2.48 (s, 3H), 2.37 (s, 3H).

### Example No. I-41:

¹H-NMR (400 MHz, CDCl₃ δ, ppm) 8.59 (s, 2H), 7.07 (s, 1H), 6.76 (t, J=55 Hz, 1H), 6.74 (s, 1H), 2.49 (s, 3H), 2.47 (s, 3H).

### Example No. I-43:

¹H-NMR (400 MHz, CDCl₃ δ, ppm) 8.19 (dd, 1H), 7.70 (dd, 1H), 7.00-6.97 (m, 2H), 6.78 (t, J=55 Hz, 1H), 6.72 (s, 1H), 2.46 (s, 3H), 2.44 (s, 3H).

### Example No. I-45:

¹H-NMR (400 MHz, CDCl₃ δ, ppm) 8.79 (s, 2H), 7.13 (s, 1H), 6.75 (t, J=55 Hz, 1H), 6.71 (s, 1H), 2.52 (s, 3H), 2.49 (s, 3H).

### Example No. I-46:

¹H-NMR (400 MHz, CDCl₃ δ, ppm) 8.81 (s, 2H), 7.15 (s, 1H), 6.75 (t, J=55 Hz, 1H), 6.69 (s, 1H), 2.53 (s, 3H), 2.48 (s, 3H).

### Example No. 1-178:

¹H-NMR (400 MHz, CDCl₃ δ, ppm) 8.41 (s, 2H), 7.03 (s, 1H), 6.20 (s, 1H), 2.47 (s, 3H), 2.45 (s, 3H), 1.99-1.95 (m, 1H), 1.04-0.99 (m, 2H), 0.82-0.78 (m, 2H).

### Example No. I-179:

¹H-NMR (400 MHz, CDCl₃ δ, ppm) 8.47 (s, 2H), 7.04 (s, 1H), 6.18 (s, 1H), 2.48 (s, 3H), 2.46 (s, 3H), 1.99-1.95 (m, 1H), 1.04-0.99 (m, 2H), 0.82-0.78 (m, 2H).

### Example No. I-180: ¹H-NMR (400 MHz, d₆-DMSO δ, ppm) 8.27 (dd, 1H), 8.21 (d, 1H), 7.17 (s, 1H), 6.53 (s, 1H), 35 2.32 (s, 3H), 2.31 (s, 3H), 2.07-2.01 (m, 1H), 1.05-1.00 (m, 2H), 0.82-0.78 (m, 2H).

### Example No. I-200:

¹H-NMR (400 MHz, CDC13 8, ppm) 8.40 (s, 2H), 7.04 (s, 1H), 6.46 (s, 1H), 2.56 (d, 2H), 2.48 (s, 3H), 2.48 (s, 3H), 1.01-0-93 (m, 1H), 0.56-0.51 (m, 2H), 0.22-0.18 (m, 2H).

### Example No. 1-201:

11-1-NMR (400 MHz, CDC13 8, ppm) 8.47 (s, 2H), 7.06 (s, 1H), 6.44 (s, 1H), 2.56 (d, 2H), 2.50 (s, 3H), 2.48 (s, 3H), 1.01-0-93 (m, 1H), 0.56-0.51 (m, 2H), 0.21-0.17 (m, 2H).

### Example No. I-202:

¹H-NMR (400 MHz, CDCl₃ δ, ppm) 8.04 (d, 1H), 7.52 (dd, 1H), 6.93 (s, 1H), 6.52 (s, 1H), 2.61 (d, 2H), 2.47 (s, 3H), 2.38 (s, 3H), 1.05-1-01 (m, 1H), 0.59-0.54 (m, 2H), 0.26-0.22 (m, 2H).

### Example No. I-244:

¹H-NMR (400 MHz, CDC13 8, ppm) 8.35 (s, 2H), 7.62 (dd, 1H), 7.55 (dd, 1H), 7.08 (s, 1H), 15 6.32 (dd, 1H), 2.52 (s, 3H), 2.19 (s, 3H).

### Example No. I-245:

¹H-NMR (400 MHz, CDCl₃ δ, ppm) 8.42 (s, 2H), 7.62 (dd, 1H), 7.54 (dd, 1H), 7.10 (s, 1H), 6.32 (dd, 1H), 2.53 (s, 3H), 2.20 (s, 3H).

### Example No. I-277:

¹H-1-NMR (400 MHz, CDCl₃ δ, ppm) 8.39 (s, 2H), 7.59 (s, 2H), 7.07 (s, 1H), 2.50 (s, 3H), 2.20 (s, 3H).

### Example No. 1-278:

¹H-NMR (400 MHz, CDCl₃ δ, ppm) 8.46 (s, 2H), 7.59-7.57 (m, 2H), 7.08 (s, 1H), 2.51 (s, 3H), 2.20 (s, 3H).

### Example No. I-288:

¹H-NMR (400 MHz, CDC13 8, ppm) 8.11 (d, 1H), 7.80 (d, 1H), 6.97 (s, 1H), 6.81 (s, 1H), 6.79 (t, J=55 Hz, 1H), 2.48 (s, 3H), 2.41 (s, 3H).

### Example No. I-289:

¹H-NMR (400 MHz, CDCl₃ δ, ppm) 8.30 (d, 1H), 7.78 (dd, 1H), 7.03 (s, 1H), 6.80 (s, IH), 6.80 (t, J=55 Hz, 1H), 2.47 (s, 3H), 2.43 (s, 3H).

### Example No. I-290:

¹H-NMR (400 MHz, CDCl₃ δ, ppm) 8.35 (d, 1H), 7.99 (d, 1H), 7.06 (s, 1H), 6.79 (t, J=55 Hz, 1H), 6.77 (s, 1H), 2.47 (s, 3H), 2.46 (s, 3H).

### Example No. I-291:

¹H-NMR (400 MHz, CDCl₃ δ, ppm) 8.44 (d, 1H), 8.12 (d, 1H), 7.06 (s, 1H), 6.79 (t, J=55 Hz, 1H), 6.77 (s, 1H), 2.47 (s, 3H), 2.46 (s, 3H).

### Example No. I-292:

¹H-NMR (400 MHz, CDCl₃ δ, ppm) 8.48 (dd, 1H), 7.96 (dd, 1H), 7.11 (dd, 1H), 7.08 (s, 1H), 10 6.76 (t, J=55 Hz, 1H), 6.63 (s, 1H), 2.49 (s, 3H), 2.46 (s, 3H).

15

### Example No. I-293:

¹H-NMR (400 MHz, CDCl₃ δ, ppm) 8.10 (d, 1H), 7.50-7.45 (m, 1H), 7.02 (dd, 1H), 6.95 (s, 1H), 6.78 (t, J=55 Hz, 1H), 6.76 (s, 1H), 2.46 (s, 3H), 2.42 (s, 3H).

### Example No. I-294:

¹H-NMR (400 MHz, CDCl₃ δ, ppm) 8.38 (s, 2H), 7.63-7.61 (m, 2H), 7.06 (s, 1H), 2.50 (s, 3H), 2.19 (s, 3H).

### Example No. 1-295:

¹H-1-NMR (400 MHz, CDCl₃ δ, ppm) 8.46 (s, 2H), 7.63 (d, 1H), 7.59 (d, 1H), 7.08 (s, 1H), 2.51 (s, 3H), 2.20 (s, 3H).

### Example No. I-296:

¹H-NMR (400 MHz, CDCl₃ δ, ppm) 8.55 (s, 2H), 7.05 (s, 1H), 6.18 (s, 1H), 2.49 (s, 3H), 2.46 (s, 3H), 1.99-1.95 (m, 1H), 1.04-0.99 (m, 2H), 0.82-0.78 (m, 2H).

### Example No. I-297:

¹H-NMR (400 MHz, CDCl₃ δ, ppm) 8.46 (s, 2H), 7.05 (s, 1H), 6.18 (s, IH), 2.49 (s, 3H), 2.46 (s, 3H), 1.44 (s, 3H), 0.98-0.95 (m, 2H), 0.84-0.81 (m, 2H).

### Example No. I-298:

¹H-NMR (400 MHz, CDCl₃ δ, ppm) 8.55 (s, 2H), 7.05 (s, 1H), 6.18 (s, 1H), 2.49 (s, 3H), 2.46 (s, 3H), 1.44 (s, 3H), 0.98-0.95 (m, 2H), 0.85-0.81 (m, 2H).

### Example No. I-299:

¹H-NMR (400 MHz, CDCl₃ δ, ppm) 8.41 (s, 2H), 7.03 (s, 1H), 6.20 (s, 1H), 2.47 (s, 3H), 2.46 (s, 3H), 1.45 (s, 3H), 0.99-0.96 (m, 2H), 0.84-0.81 (m, 2H).

### Example No. I-300:

¹H-1-NMR (400 MHz, CDC13 8, ppm) 8.55 (s, 2H), 7.06 (s, 1H), 6.43 (s, 1H), 2.56 (d, 2H), 2.50 (s, 3H), 2.48 (s, 3H), 1.01-0-93 (m, 1H), 0.56-0.51 (m, 2H), 0.21-0.17 (m, 2H).

### Example No. I-301:

¹H-NMR (400 MHz, CDC13 5, ppm) 8.05 (d, 1H), 7.53 (dd, 1H), 6.91 (s, 1H), 6.26 (s, 1H), 2.45 (s, 3H), 2.37 (s, 3H), 1.49 (s, 3H), 1.05-1.02 (m, 2H), 0.86-0.83 (m, 2H).

The present invention further provides for the use of one or more compounds of the general formula (I) and/or salts thereof, as defined above, preferably in one of the embodiments identified as preferred or particularly preferred, in particular one or more compounds of the formulae (1-1) to (1-301) and/or salts thereof, in each case as defined above, as herbicide and/or plant growth regulator, preferably in crops of useful plants and/or ornamentals.

The present invention further provides a method of controlling harmful plants and/or for regulating the growth of plants, characterized in that an effective amount
of one or more compounds of the general formula (I) and/or salts thereof, as defined above, preferably in one of the embodiments identified as preferred or particularly preferred, in particular one or more compounds of the formulae (I-1) to (I-301) and/or salts thereof, in each case as defined above, or
of a composition of the invention, as defined below, is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I-1) to (I-301) and/or salts thereof, in each case as defined above, or
of a composition of the invention, as defined below, is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop-growing land or non-crop-growing land) or the area under cultivation (i.e. the area on which the unwanted plants will grow).

The present invention also further provides a method for controlling for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the formulae (I-1) to (I-301) and/or salts thereof, in each case as defined above, or
of a composition of the invention, as defined below, is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the inventive compounds or the inventive compositions can be applied for example by pre-sowing (if appropriate also by incorporation into the soil), pre-emergence and/or post-emergence processes. Specific examples of some representatives of the monocotyledonous and dicotyledonous weed flora which can be controlled by the inventive compounds are as follows, though there is no intention to restrict the enumeration to particular species.

In a method of the invention for controlling harmful plants or for regulating the growth of plants, preference is given to using one or more compounds of the general formula (I) and/or salts thereof for control of harmful plants or for regulation of growth in crops of useful plants or ornamental plants, where the useful plants or ornamental plants in a preferred configuration are transgenic plants.

The inventive compounds of the general formula (I) and/or salts thereof are suitable for controlling the following genera of monocotyledonous and dicotyledonous harmful plants:

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium,

Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindemia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the inventive compounds of the general formula (I) are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

If the active ingredients of the general formula (I) are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the inventive compounds of the general formula (I) display outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective inventive compound and its application rate. For these reasons, the present compounds are very suitable for selective control of unwanted plant growth in plant crops such as agriculturally useful plants or ornamental plants.

In addition, the inventive compounds of the general formula (I) (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the active ingredients of the general formula (I) can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the inventive compounds of the general formula (I) and/or salts thereof in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferable to employ the inventive compounds of the general formula (I) also as herbicides in crops of useful plants which are resistant, or have been made resistant by recombinant means, to the phytotoxic effects of the herbicides.

By virtue of their herbicidal and plant growth regulatory properties, the inventive compounds of the general formula (I) can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents.

For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the inventive compounds of the general formula (I) or salts thereof in economically important transgenic crops of useful plants and ornamentals, for example of cereals such as wheat, barley, rye, oats, triticale, millet, rice, cassava and corn, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Sequences of this kind are known to the person skilled in the art (see, for example, Braun et al., EMBO J. 11 (1992), 3219-3227). The nucleic acid molecules can also be expressed in the organelles of the plant cells. The transgenic plant cells can be regenerated by known techniques to give rise to entire plants. In principle, the transgenic plants may be plants of any desired plant species, i.e. not only monocotyledonous but also dicotyledonous plants.

Obtainable in this way are transgenic plants having properties altered by overexpression, suppression or inhibition of homologous (=natural) genes or gene sequences or expression of heterologous (=foreign) genes or gene sequences.

It is preferable to employ the inventive compounds of the general formula (I) in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active ingredients.

When the inventive compounds of the general formula (I) are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the general formula (I) and/or salts thereof as herbicides for controlling harmful plants in crops of useful plants or ornamentals, optionally in transgenic crop plants.

Preference is given to the use of compounds of the general formula (I) in cereals, here preferably corn, wheat, barley, rye, oats, millet or rice, by the pre- or post-emergence method.

Preference is also given to the use of compounds of the general formula (I) in soybean by the pre-emergence or post-emergence method.

The use of inventive compounds of the formula (I) for the control of harmful plants or for growth regulation of plants also includes the case in which a compound of the general formula (I) or its salt is not formed from a precursor substance (“prodrug”) until after application on the plant, in the plant or in the soil.

The invention also provides the use of one or more compounds of the general formula (I) or salts thereof or of a composition according to the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises
(a) one or more compounds of the general formula (I) and/or salts thereof, as defined above, preferably in one of the embodiments identified as preferred or particularly preferred, in particular one or more compounds of the formulae (I-1) to (I-301) and/or salts thereof, in each case as defined above,
and
(b) one or more further substances selected from groups (i) and/or (ii):
(i) one or more further agrochemically active substances, preferably selected from the group consisting of insecticides, acaricides, nematicides, further herbicides (i.e. those not conforming to the general formula (I) defined above), fungicides, safeners, fertilizers and/or further growth regulators,
(ii) one or more formulation auxiliaries customary in crop protection.

The further agrochemically active substances of component (i) of a composition of the invention are preferably selected from the group of substances mentioned in “The Pesticide Manual”, 16th edition, The British Crop Protection Council and the Royal Soc. of Chemistry, 2012.

A herbicidal or plant growth-regulating composition of the invention comprises preferably one, two, three or more formulation auxiliaries (ii) customary in crop protection selected from the group consisting of surfactants, emulsifiers, dispersants, film formers, thickeners, inorganic salts, dusting agents, carriers that are solid at 25° C. and 1013 mbar, preferably adsorptive granulated inert materials, wetting agents, antioxidants, stabilizers, buffer substances, antifoam agents, water, organic solvents, preferably organic solvents miscible with water in any ratio at 25° C. and 1013 mbar.

The inventive compounds of the general formula (I) can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The inventive compounds of the general formula (I) and/or salts thereof can be formulated in various ways according to which biological and/or physicochemical parameters are specified. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation auxiliaries, such as inert materials, surfactants, solvents and further additives, are known to the person skilled in the art and are described, for example, in: Watkins, “Handbook of Insecticide Dust Diluents and Carriers”, 2nd ed., Darland Books, Caldwell N.J., H.v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesellschaft, Stuttgart 1976, Winnacker-Küchler, “Chemische Technologie”, Volume 7, C. Hanser Verlag Munich, 4th ed. 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules - if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London; J.E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G.C. Klingman, “Weed Control as a Science”, John Wiley and Sons, Inc., New York, 1961, pages 81-96 and J.D. Freyer, S.A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of 0.1 to 99% by weight, preferably 0.5 to 95% by weight, more preferably 1 to 90% by weight, especially preferably 2 to 80% by weight, of active ingredients of the general formula (I) and salts thereof.

In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partly on whether the active compound is in liquid or solid form and on which granulation auxiliaries, fillers, and so forth are used. In the water-dispersible granules, the content of active ingredient is, for example, between 1% and 95% by weight, preferably 10 between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described, inter alia, in “Chemistry and Technology of Agrochemical Formulations”, ed. D. A. Knowles, Kluwer Academic Publishers (1998).

The inventive compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tankmix. The combination formulations can be produced on the basis of the abovementioned formulations, taking account of the physical properties and stabilities of the active ingredients to be combined.

Combination partners usable for the inventive compounds of the general formula (I) in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012, and the literature cited therein.

Of particular interest is the selective control of harmful plants in crops of useful plants and ornamentals. Although the inventive compounds of the general formula (I) have already demonstrated very good to adequate selectivity in a large number of crops, in principle, in some crops and in particular also in the case of mixtures with other, less selective herbicides, phytotoxicities on the crop plants may occur. In this connection, combinations of inventive compounds (I) that are of particular interest are those which comprise the compounds of the general formula (I) or their combinations with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide formulations or herbicide-safener formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The application rate of the compounds of the general formula (I) and/or their salts is affected to a certain extent by external conditions such as temperature, humidity, etc. The application rate may vary within wide limits. For the application as a herbicide for controlling harmful plants, the total amount of compounds of the general formula (I) and their salts is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to pre-emergence and to post-emergence application.

When the inventive compounds of the general formula (I) and/or salts thereof are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to pre-emergence and to post-emergence application.

The application as culm stabilizer may take place at various stages of the growth of the plants. Preferred is, for example, the application after the tillering phase, at the beginning of the longitudinal growth. As an alternative, application as plant growth regulator is also possible by treating the seed, which includes various techniques for dressing and coating seed. The application rate depends on the particular techniques and can be determined in preliminary tests.

Combination partners usable for the inventive compounds of the general formula (I) in compositions of the invention (e.g. mixed formulations or in a tankmix) are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as described, for example, from Weed Research 26 (1986) 441-445 or “The Pesticide Manual”, 16th edition, The British Crop Protection Council and the Royal Soc. of Chemistry, 2012, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Examples of such herbicidal mixing partners are:
acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-12-chloro-4-fluoro-5-14-(3-fluoropropyl)-4,5-dihydro-5-oxo-1H-tetrazol-1-yl1-phenyflethanesulfonamide, F-7967, i.e. 3-[7-chloro-5-fluoro-2-(trifluoromethyl)-1H-benzimidazol-4-yl]-1-methyl-6-(trifluoromethyppyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl) O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, Imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-y]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 147-fluoro-3-oxo-4-(prop-2-yn-1-yl)-3,4-dihydro-2H-1,4-benzoxazin-6-yl1-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{24(4,6-dimethoxypyrimidin-2-ypoxylbenzyl}aniline, and the following compounds:

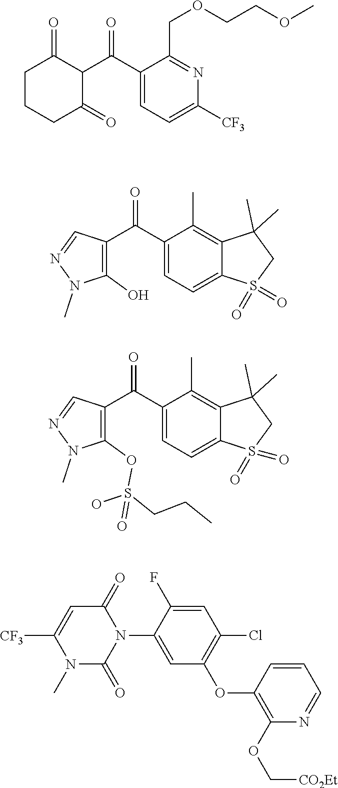

Examples of plant growth regulators as possible mixing partners are:
acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the inventive compounds of the general formula (I) also include, for example, the following safeners:

S1) Compounds from the group of heterocyclic carboxylic acid derivatives:

S1^{a}) Compounds of the dichlorophenylpyrazoline-3-carboxylic acid type (S1^{a}), preferably compounds such as
1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;

S1^{b}) Derivatives of dichlorophenylpyrazolecarboxylic acid (Sib), preferably compounds such as ethyl 1-(2,4-dichlorophenyl)-5-methylpyrazole-3-carboxylate (S1^{b}), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A- 333131 and EP-A-269806;

S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;

S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;

S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897

S2) Compounds from the group of the 8-quinolinoxy derivatives (S2):

S2^{a}) Compounds of the 8-quinolinoxyacetic acid type (Sr), preferably 1-methylhexyl (5-chloro-8-quinolinoxy)acetate (“cloquintocet-mexyl”) (S2-1), 1,3-dimethylbut-l-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5),

methyl (5-chloro-8-quinolinoxy)acetate (S2-6),

allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5- chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminium, iron, ammonium, quaternary ammonium, sulfonium or phosphonium salts thereof, as described in WO-A-2002/34048;

S2^{b}) Compounds of the (5-chloro-8-quinolinoxy)malonic acid type (Sr), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.

S3) Active ingredients of the dichloroacetamide type (S3), which are frequently used as pre-emergence safeners (soil-acting safeners), for example

“dichlormid” (N,N-diallyl-2,2-dichloroacetamide) (S3-1),

“R-29148” (3-dichloroacetyl-2,2,5-trimethyl-1,3-oxazolidine) from Stauffer (S3-2),

“R-28725” (3-dichloroacetyl-2,2-dimethyl-1,3-oxazolidine) from Stauffer (S3-3),

“benoxacor” (4-dichloroacetyl-3,4-dihydro-3-methyl-2H-1,4-benzoxazine) (S3-4),

“PPG-1292” (N-allyl-N-[(1,3-dioxolan-2-yOmethyl]dichloroacetamide) from PPG Industries (S3-5),

“DKA-24” (N-allyl-N-Rallylaminocarbonyl)methylklichloroacetamide) from Sagro-Chem (S3-6),

“AD-67” or “MON 4660” (3-dichloroacetyl-1-oxa-3-azaspiro[4.5]decane) from Nitrokemia or Monsanto (S3-7),

“TI-35” (1-dichloroacetylazepane) from TRI-Chemical RT (S3-8), “diclonon” (dicyclonon) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10), and the (R) isomer thereof (S3-11).

S4) Compounds from the class of the acylsulfonamides (S4):

S4^{a}) N-Acylsulfonamides of the formula (S4a) and salts thereof, as described in WO-A-97/45016,

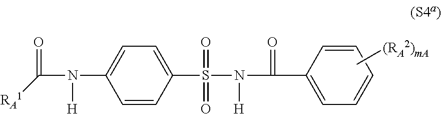

in which
R_{A}¹ is (C₁-C₆)-alkyl, (C₃-C₆)-cycloalkyl, where the 2 latter radicals are substituted by vA substituents from the group of halogen, (CI-CO-alkoxy, (C₁-C₄)-haloalkoxy and (C₁-C₄)-alkylthio and, in the case of cyclic radicals, also by (C₁-C₄)-alkyl and (C₁-C₄)-haloalkyl;
R_{A}² is halogen, (C₁-C₄)-alkyl, (C₁-C₄)-alkoxy, CF₃;
m_{A} is 1 or 2;
v_{A} is 0, 1, 2 or 3;

S4^{b}) Compounds of the 4-(benzoylsulfamoyl)benzamide type of the formula (S₄^{b}) and salts thereof, as described in WO-A-99/16744,

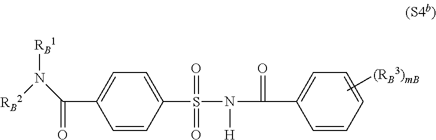

in which
R_{B}¹, R_{B}² are independently hydrogen, (C₁-C₆)-alkyl, (C₃-C₆)-cycloalkyl, (C₃-C₆)-alkenyl, (C₃-C₆)-alkynyl,
R_{B}³ is halogen, (C₁-C₄)-alkyl, (C₁-C₄)-haloalkyl or (C₁-C₄)-alkoxy and
m_{B} is 1 or 2,
e.g. those in which
R_{B}¹=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-C1-2-0Me (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (RR')=2-OMe (S4-5);

S4^{c}) Compounds from the class of the benzoylsulfamoylphenylureas of the formula (S4^{c}), as described in EP-A-365484,

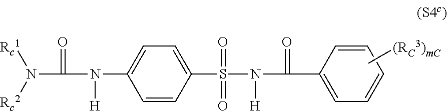

in which
R_{C}¹, R_{C}² are independently hydrogen, (C₁-C₈)-alkyl, (C₃-C₈)-cycloalkyl, (C₃-C₆)-alkenyl, (C₃-C₆)-alkynyl,
R_{C}³ is halogen, (C₁-C₄)-alkyl, (C₁-C₄)-alkoxy, CF₃ and
m_{C} is 1 or 2;
for example
1-[4-(N-2-methoxybenzoylsulfamoyl)phenyl]-3-methylurea,
1-[4-(N-2-methoxybenzoylsulfamoyflphenyl]-3,3-dimethylurea,
1-[4-(N-4,5-dimethylbenzoylsulfamoyl)phenyl]-3-methylurea;

S4^{d}) Compounds of the N-phenylsulfonylterephthalamide type of the formula (S4^{d}) and salts thereof, which are known, for example, from CN 101838227,

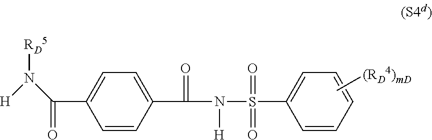

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active ingredients from the class of the hydroxyaromatics and the aromatic-aliphatic carboxylic acid derivatives (S5), for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.

S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.

S8) Compounds of the formula (S8), as described in WO-A-98/27049,

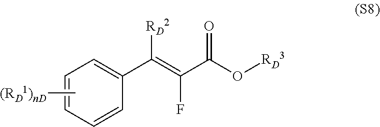

in which the symbols and indices are defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,

R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof,

n_{D} is an integer from 0 to 2.

S9) Active ingredients from the class of the 3-(5-tetrazolylcarbonyl)-2-quinolones (S9), for example 1,2-dihydro-4-hydroxy-1 -ethyl-3-(5-tetrazolylcarbonyl)-2-quinolone (CAS Reg. No.: 219479-18-2), 1,2-dihydro-4-hydroxy-l-methyl-3-(5-tetrazolylcarbonyl)-2-quinolone (CAS Reg. No. 95855-00-8), as described in WO-A-1999/000020.

S10) Compounds of the formulae (S10^{a}) or (S 10^{b}) as described in WO-A-2007/023719 and WO-A-2007/023764,

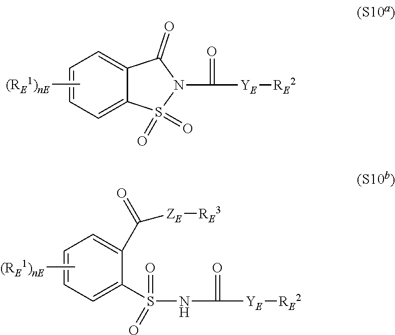

in which

R_{E}¹ is halogen, methoxy, nitro, cyano, CF₃, OCF₃,

Y_{E}, Z_{E} are independently O or S,

nE is an integer from 0 to 4,

R_{E}² is (C₁-C₁₆)-alkyl, (C₂-C₆)-alkenyl, (C₃-C₆)-cycloalkyl, aryl; benzyl, halobenzyl,

R_{E}³ is hydrogen or (C₁-C₆)-alkyl.

S11) Active ingredients of the oxyimino compounds type (S11), which are known as seed-dressing agents, for example

“oxabetrinil” ((Z)-1,3-dioxolan-2-ylmethoxyimino(phenyl)acetonitrile) (S11-1), which is known as a seed-dressing safener for millet/sorghum against metolachlor damage, “fluxofenim” (1-(4-chlorophenyl)-2,2,2-trifluoro-l-ethanone O-(1,3-dioxolan-2-ylmethyl)oxime) (S11-2), which is known as a seed-dressing safener for millet/sorghum against metolachlor damage, and
“cyometrinil” or “CGA-43089” ((Z)-cyanomethoxyimino(phenyl)acetonitrile) (S11-3), which is known as a seed-dressing safener for millet/sorghum against metolachlor damage.

S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.

S13) One or more compounds from group (S13):
“naphthalic anhydride” (1,8-naphthalenedicarboxylic anhydride) (S13-1), which is known as a seed-dressing safener for maize against thiocarbamate herbicide damage,
“fenclorim” (4,6-dichloro-2-phenylpyrimidine) (S13-2), which is known as a safener for pretilachlor in sown rice,
“flurazole” (benzyl 2-chloro-4-trifluoromethyl-1,3-thiazole-5-carboxylate) (S13-3), which is known as a seed-dressing safener for millet/sorghum against alachlor and metolachlor damage, “CL 304415” (CAS Reg. No. 31541-57-8)
(4-carboxy-3,4-dihydro-2H-l-benzopyran-4-acetic acid) (S13-4) from American Cyanamid, which is known as a safener for corn against damage by imidazolinones,
“MG 191” (CAS Reg. No. 96420-72-3) (2-dichloromethyl-2-methyl-1,3-dioxolane) (S13-5) from Nitrokemia, which is known as a safener for maize,
“MG 838” (CAS Reg. No. 133993-74-5)
(2-propenyl 1-oxa-4-azaspiro[4.5]decane-4-carbodithioate) (S13-6) from Nitrokemia
“disulfoton” (0,0-diethyl S-2-ethylthioethyl phosphorodithioate) (S13-7),
“dietholate” (0,0-diethyl 0-phenyl phosphorothioate) (S13-8),
“mephenate” (4-chlorophenyl methylcarbamate) (S13-9).

S14) Active ingredients which, in addition to herbicidal action against harmful plants, also have safener action on crop plants such as rice, for example

“dimepiperate” or “MY-93” (S-1-methyl 1-phenylethylpiperidine-l-carbothioate), which is known as a safener for rice against damage by the herbicide molinate,
“daimuron” or “SK 23” (1-(1-methyl-l-phenylethyl)-3-p-tolylurea), which is known as a safener for rice against damage by the herbicide imazosulfuron,
“cumyluron”=“JC-940” (3(2-chlorophenylmethyl)-1-(1-methyl-1-phenylethypurea, see JP-A-60087270), which is known as a safener for rice against damage by some herbicides,
“methoxyphenone” or “NK 049” (3,3′-dimethyl-4-methoxybenzophenone), which is known as a safener for rice against damage by some herbicides,
“CSB” (1-bromo-4-(chloromethylsulfonyl)benzene) from Kumiai, (CAS Reg. No. 54091-06-4), which is known as a safener against damage by some herbicides in rice.

S15) Compounds of the formula (S15) or tautomers thereof

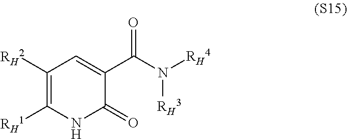

as described in WO-A-2008/131861 and WO-A-2008/131860
in which

R_{H}¹ is a (C,-C6)-haloalkyl radical and

R_{H}² is hydrogen or halogen and

R_{H}³, R_{H}⁴ are independently hydrogen, (C1-C16)-alkyl, (C2-C16)-alkenyl or (C2-C16)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, RCI-C4)-alkoxylcarbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or (C₃-C₆)-cycloalkyl, (C4-C6)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring, where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, alkylamino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or

R_{H}¹ is (C₁-C₄)-alkoxy, (C₂-C₄)-alkenyloxy, (C₂-C₆)-alkynyloxy or (C₂-C₄)-haloalkoxy and

R_{H}² is hydrogen or (C₁-C₄)-alkyl or

R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.

S16) Active ingredients which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl3,6-dichloro-2-methoxybenzoate (lactidichloro-ethyl).

Preferred safeners in combination with the inventive compounds of the general formula (I) and/or salts thereof, especially with the compounds of the formulae (I-1) to (I-301) and/or salts thereof, are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### Biological Examples

### A. Post-Emergence Herbicidal Efficacy at 320 g/ha

Seeds of mono- and dicotyledonous weed plants were placed in plastic pots in sandy loam soil (doubly sown with in each case one species of mono- or dicotyledonous weed plants per pot), covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were applied onto the green parts of the plants as aqueous suspension or emulsion with addition of 0.5% additive at a water application rate of 600 liters per hectare (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables A1 to A 11 below show the effects of selected compounds of the general formula (I) according to Table 1 on various harmful plants and an application rate corresponding to 320 g/ha, which were obtained by the experimental procedure mentioned above.

TABLE A1
Post-emergence efficacy against Abutilon theophrasti (ABUTH)

| Example number | Dosage [g/ha] | ABUTH |
|---|---|---|
| I-1 | 320 | 90 |
| I-3 | 320 | 90 |
| I-6 | 320 | 90 |
| I-8 | 320 | 90 |
| I-14 | 320 | 90 |
| I-41 | 320 | 90 |
| I-43 | 320 | 90 |
| I-45 | 320 | 90 |
| I-178 | 320 | 90 |
| I-179 | 320 | 90 |
| I-201 | 320 | 90 |
| I-277 | 320 | 90 |
| I-292 | 320 | 90 |
| I-294 | 320 | 90 |
| I-298 | 320 | 90 |
| I-299 | 320 | 90 |

TABLE A2
Post-emergence efficacy against
Alopecurus myosuroides (ALOMY)

| Example number | Dosage [g/ha] | ALOMY |
|---|---|---|
| I-1 | 320 | 90 |
| I-2 | 320 | 90 |
| I-5 | 320 | 100 |
| I-7 | 320 | 90 |
| I-8 | 320 | 100 |
| I-11 | 320 | 90 |
| I-14 | 320 | 100 |
| I-43 | 320 | 90 |

TABLE A3
Post-emergence efficacy against Amaranthus retroflexus (AMARE)

| Example number | Dosage [g/ha] | AMARE |
|---|---|---|
| I-1 | 320 | 100 |
| I-2 | 320 | 100 |
| I-3 | 320 | 90 |
| I-5 | 320 | 100 |
| I-7 | 320 | 90 |
| I-8 | 320 | 100 |
| I-11 | 320 | 100 |
| I-13 | 320 | 90 |
| I-14 | 320 | 90 |
| I-41 | 320 | 100 |
| I-43 | 320 | 100 |
| I-45 | 320 | 100 |
| I-46 | 320 | 100 |
| I-178 | 320 | 100 |
| I-179 | 320 | 90 |
| I-200 | 320 | 90 |
| I-277 | 320 | 100 |
| I-278 | 320 | 100 |
| I-288 | 320 | 100 |
| I-289 | 320 | 100 |
| I-290 | 320 | 100 |
| I-291 | 320 | 90 |
| I-292 | 320 | 100 |
| I-294 | 320 | 100 |
| I-295 | 320 | 90 |
| I-296 | 320 | 90 |

TABLE A4
Post-emergence efficacy against Digitaria sanguinalis (DIGSA)

| Example number | Dosage [g/ha] | DIGSA |
|---|---|---|
| I-3 | 320 | 90 |
| I-5 | 320 | 90 |
| I-10 | 320 | 100 |

TABLE A5
Post-emergence efficacy against Echinochloa crus-galli (ECHCG)

| Example number | Dosage [g/ha] | ECHCG |
|---|---|---|
| I-7 | 320 | 90 |
| I-43 | 320 | 90 |

TABLE A6
Post-emergence efficacy against Bassia scoparia (KCHSC)

| Example number | Dosage [g/ha] | KCHSC |
|---|---|---|
| I-1 | 320 | 90 |
| I-3 | 320 | 100 |
| I-5 | 320 | 90 |
| I-6 | 320 | 90 |
| I-8 | 320 | 90 |
| I-11 | 320 | 90 |
| I-14 | 320 | 100 |
| I-41 | 320 | 100 |
| I-45 | 320 | 90 |
| I-178 | 320 | 90 |
| I-179 | 320 | 90 |
| I-277 | 320 | 100 |
| I-292 | 320 | 90 |
| I-295 | 320 | 90 |

TABLE A7
Post-emergence efficacy against Poa annua (POAAN)

| Example number | Dosage [g/ha] | POAAN |
|---|---|---|
| I-1 | 320 | 90 |
| I-3 | 320 | 100 |
| I-6 | 320 | 90 |
| I-10 | 320 | 100 |
| I-41 | 320 | 100 |
| I-43 | 320 | 90 |
| I-45 | 320 | 100 |
| I-46 | 320 | 90 |
| I-178 | 320 | 100 |
| I-179 | 320 | 90 |
| I-202 | 320 | 100 |
| I-277 | 320 | 90 |
| I-288 | 320 | 90 |
| I-289 | 320 | 90 |
| I-292 | 320 | 100 |
| I-294 | 320 | 90 |
| I-296 | 320 | 90 |

TABLE A8
Post-emergence action against Stellaria media (STEME)

| Example number | Dosage [g/ha] | STEME |
|---|---|---|
| I-1 | 320 | 100 |
| I-2 | 320 | 100 |
| I-3 | 320 | 90 |
| I-5 | 320 | 90 |
| I-7 | 320 | 100 |
| I-8 | 320 | 90 |
| I-11 | 320 | 100 |
| I-14 | 320 | 100 |
| I-41 | 320 | 100 |
| I-43 | 320 | 100 |
| I-45 | 320 | 100 |
| I-46 | 320 | 100 |
| I-178 | 320 | 100 |
| I-179 | 320 | 100 |
| I-180 | 320 | 100 |
| I-200 | 320 | 90 |
| I-201 | 320 | 90 |
| I-202 | 320 | 90 |
| I-277 | 320 | 90 |
| I-278 | 320 | 90 |
| I-288 | 320 | 90 |
| I-289 | 320 | 90 |
| I-292 | 320 | 100 |
| I-294 | 320 | 100 |
| I-295 | 320 | 90 |
| I-296 | 320 | 100 |
| I-298 | 320 | 90 |
| I-299 | 320 | 90 |
| I-300 | 320 | 90 |
| I-301 | 320 | 90 |

TABLE A9
Post-emergence efficacy against Veronica persica (VERPE)

| Example number | Dosage [g/ha] | VERPE |
|---|---|---|
| I-1 | 320 | 100 |
| I-2 | 320 | 100 |
| I-3 | 320 | 100 |
| I-5 | 320 | 100 |
| I-7 | 320 | 100 |
| I-8 | 320 | 100 |
| I-10 | 320 | 90 |
| I-11 | 320 | 100 |
| I-14 | 80 | 100 |
| I-41 | 320 | 100 |
| I-43 | 320 | 100 |
| I-45 | 320 | 100 |
| I-46 | 320 | 100 |
| I-178 | 320 | 100 |
| I-179 | 320 | 100 |
| I-180 | 320 | 90 |
| I-200 | 320 | 90 |
| I-201 | 320 | 90 |
| I-277 | 320 | 90 |
| I-278 | 320 | 90 |
| I-288 | 320 | 90 |
| I-289 | 320 | 90 |
| I-290 | 320 | 90 |
| I-291 | 320 | 90 |
| I-292 | 320 | 100 |
| I-294 | 320 | 100 |
| I-295 | 320 | 100 |
| I-296 | 320 | 100 |
| I-297 | 320 | 90 |
| I-298 | 320 | 90 |
| I-299 | 320 | 90 |
| I-301 | 320 | 90 |

TABLE A10
Post-emergence efficacy against Lolium rigidum (LOLRI)

| Example number | Dosage [g/ha] | LOLRI |
|---|---|---|
| I-41 | 320 | 90 |

TABLE A11
Post-emergence efficacy against Setaria viridis (SETVI)

| Example number | Dosage [g/ha] | SETVI |
|---|---|---|
| I-41 | 320 | 90 |

As shown by way of example by the results in tables A 1 -A11, inventive compounds No. I-1, I-2, I-3, I-5, I-6, I-7, I-8, I-10, I-11, I-13, I-14, I-41, I-43, I-45, I-46, I-178, I-179, I-180, I-200, I-201, I-202, I-277, I-278, I-288, I-289, I-290, I-291, I-292, I-294, I-295, I-296, I-297,

I-298, I-299, I-300 and I-301 in the case of post-emergence treatments have very good herbicidal efficacy against the following harmful plants at an application rate of 320 g of active substance per hectare: Abutilon theophrasti (ABUTH), Alopecurus tnyosuroides (ALOMY), Amaranthus retroflexus (AMARE), Digitaria sanguinalis (DIGSA), Echinochloa crus-galli (ECHCG), Bassin scoparia (KCHSC), Lolium rigidum (LOLRI), Poa annua (POAAN), Setaria viridis (SETVI), Stellaria media (STEME) and Veronica persica (VERPE).

### B. Post-Emergence Herbicidal Efficacy at 80 g/ha

Seeds of monocotyledonous and dicotyledonous weeds and crop plants were placed in sandy loam in plastic or organic planting pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 600 l/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables B1 to B6 below show the effects of selected compounds of the general formula (I) according to Table 1 on various harmful plants and an application rate corresponding to 80 g/ha, which were obtained by the experimental procedure mentioned above.

TABLE B1
Post-emergence efficacy against Alopecurus myosuroides (ALOMY)

| Example | Dosage | |
|---|---|---|
| number | [g/ha] | ALOMY |
| I-2 | 80 | 90 |
| I-3 | 80 | 90 |
| I-4 | 80 | 90 |
| I-5 | 80 | 80 |
| I-7 | 80 | 90 |
| I-10 | 80 | 80 |

TABLE B2
Post-emergence efficacy against Amaranthus retroflexus (AMARE)

| Example | Dosage | |
|---|---|---|
| number | [g/ha] | AMARE |
| I-1 | 80 | 100 |
| I-2 | 80 | 100 |
| I-3 | 80 | 90 |
| I-4 | 80 | 100 |
| I-5 | 80 | 100 |
| I-7 | 80 | 80 |
| I-8 | 80 | 100 |
| I-14 | 80 | 100 |
| I-41 | 80 | 90 |
| I-45 | 80 | 80 |
| I-277 | 80 | 80 |
| I-292 | 80 | 90 |
| I-293 | 80 | 100 |
| I-294 | 80 | 80 |

TABLE B3
Post-emergence efficacy against Polygonum convolvulus (POLCO)

| Example | Dosage | |
|---|---|---|
| number | [g/ha] | POLCO |
| I-3 | 80 | 100 |
| I-4 | 80 | 90 |
| I-41 | 80 | 80 |
| I-293 | 80 | 100 |
| I-294 | 80 | 80 |

TABLE B4
Post-emergence efficacy against Setaria viridis (SETVI)

| Example | Dosage | |
|---|---|---|
| number | [g/ha] | SETVI |
| I-45 | 80 | 80 |
| I-277 | 80 | 80 |
| I-294 | 80 | 90 |

TABLE B5
Post-emergence efficacy against Veronica persica (VERPE)

| Example | Dosage | |
|---|---|---|
| number | [g/ha] | VERPE |
| I-3 | 80 | 80 |
| I-4 | 80 | 90 |
| I-41 | 80 | 90 |
| I-292 | 80 | 90 |

TABLE B6
Post-emergence efficacy against Viola tricolor (VIOTR)

| Example | Dosage | |
|---|---|---|
| number | [g/ha] | VIOTR |
| I-1 | 80 | 90 |
| I-2 | 80 | 90 |
| I-3 | 80 | 100 |
| I-4 | 80 | 80 |
| I-5 | 80 | 80 |
| I-7 | 80 | 90 |
| I-8 | 80 | 90 |
| I-10 | 80 | 100 |
| I-14 | 80 | 100 |
| I-43 | 80 | 80 |
| I-292 | 80 | 90 |

As shown by way of example by the results in tables B1-B6, inventive compounds No. I-1, I-2, I-3, I-4, I-5, I-7, I-8, I-10, I-14, I-41, I-43, I-45, I-277, I-292, I-293 and I-294 in the case of post-emergence treatment have very good herbicidal efficacy against the following harmful plants at an application rate of 80 g of active substance per hectare: Alopecurus myosuroides (ALOMY), Amaranthus retroflexus (AMARE), Polygonum convolvulus (POLCO), Setaria viridis (SETVI), Veronica persica (VERPE) and Viola tricolor (VIOTR).

### C. Pre-Emergence Herbicide Efficacy at 320 g/ha

Seeds of mono- and dicotyledonous weed plants were placed in plastic pots in sandy loam soil (doubly sown with in each case one species of mono- or dicotyledonous weed plants per pot) and covered with soil. The compounds of the invention, formulated in the form of wettable powders (WP) or as emulsion concentrates (EC), were then applied onto the surface of the covering soil as aqueous suspension or emulsion with addition of 0.5% additive at a water application rate of 600 liters per hectare (converted).

After the treatment, the pots were placed in a greenhouse and kept under good growth conditions for the test plants. After about 3 weeks, the effect of the preparations was scored visually in comparison with untreated controls as percentages.

For example, 100% activity=the plants have died, 0% activity=like control plants.

Tables C1 to C12 below show the effects of selected compounds of the general formula (I) according to Table 1 on various harmful plants and an application rate corresponding to 320 g/ha, which were obtained by the experimental procedure mentioned above.

TABLE C1
Pre-emergence efficacy against Abutilon theophrasti (ABUTH)

| Example | Dosage | |
|---|---|---|
| number | [g/ha] | ABUTH |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 100 |
| I-10 | 320 | 100 |
| I-11 | 320 | 90 |
| I-12 | 320 | 90 |
| I-14 | 320 | 100 |
| I-41 | 320 | 100 |
| I-43 | 320 | 100 |
| I-45 | 320 | 100 |
| I-46 | 320 | 90 |
| I-178 | 320 | 100 |
| I-179 | 320 | 100 |
| I-200 | 320 | 90 |
| I-201 | 320 | 90 |
| I-245 | 320 | 90 |
| I-277 | 320 | 100 |
| I-278 | 320 | 100 |
| I-292 | 320 | 90 |
| I-294 | 320 | 100 |
| I-295 | 320 | 100 |
| I-296 | 320 | 100 |
| I-297 | 320 | 90 |
| I-299 | 320 | 90 |

TABLE C2
Pre-emergence efficacy against Alopecurus myosuroides (ALOMY)

| Example | Dosage | |
|---|---|---|
| number | [g/ha] | ALOMY |
| I-1 | 320 | 100 |
| I-2 | 320 | 100 |
| I-3 | 320 | 100 |
| I-5 | 320 | 100 |
| I-6 | 320 | 90 |
| I-7 | 320 | 90 |
| I-8 | 320 | 100 |
| I-11 | 320 | 100 |
| I-41 | 320 | 90 |
| I-45 | 320 | 100 |

TABLE C3
Pre-emergence efficacy against Amaranthus retroflexus (AMARE)

| Example | Dosage | |
|---|---|---|
| number | [g/ha] | AMARE |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 100 |
| I-10 | 320 | 100 |
| I-11 | 320 | 100 |
| I-12 | 320 | 100 |
| I-14 | 320 | 100 |
| I-41 | 320 | 100 |
| I-43 | 320 | 100 |
| I-45 | 320 | 100 |
| I-46 | 320 | 90 |
| I-178 | 320 | 100 |
| I-179 | 320 | 100 |
| I-180 | 320 | 100 |
| I-200 | 320 | 100 |
| I-201 | 320 | 90 |
| I-202 | 320 | 90 |
| I-245 | 320 | 100 |
| I-277 | 320 | 100 |
| I-292 | 320 | 100 |
| I-294 | 320 | 100 |
| I-295 | 320 | 100 |
| I-296 | 320 | 100 |
| I-297 | 320 | 90 |
| I-298 | 320 | 90 |
| I-299 | 320 | 100 |

TABLE C4
Pre-emergence efficacy against Digitaria sanguinalis (DIGSA)

| Example | Dosage | |
|---|---|---|
| number | [g/ha] | DIGSA |
| I-2 | 320 | 100 |
| I-3 | 320 | 100 |
| I-5 | 320 | 100 |
| I-7 | 320 | 100 |
| I-8 | 320 | 100 |
| I-10 | 320 | 90 |

TABLE C5
Pre-emergence efficacy against Echinochloa crus-galli (ECHCG)

| Example | Dosage | |
|---|---|---|
| number | [g/ha] | ECHCG |
| I-1 | 320 | 100 |
| I-2 | 320 | 100 |
| I-3 | 320 | 100 |
| I-5 | 320 | 90 |
| I-6 | 320 | 100 |
| I-7 | 320 | 100 |
| I-10 | 320 | 100 |
| I-41 | 320 | 90 |
| I-43 | 320 | 90 |
| I-45 | 320 | 100 |
| I-178 | 320 | 100 |
| I-179 | 320 | 100 |
| I-200 | 320 | 90 |
| I-277 | 320 | 100 |
| I-278 | 320 | 100 |
| I-294 | 320 | 100 |
| I-295 | 320 | 90 |
| I-296 | 320 | 90 |
| I-299 | 320 | 90 |

TABLE C6
Pre-emergence efficacy against Bassia scoparia (KCHSC)

| Example | Dosage | |
|---|---|---|
| number | [g/ha] | KCHSC |
| I-1 | 320 | 90 |
| I-2 | 320 | 90 |
| I-3 | 320 | 100 |
| I-5 | 320 | 100 |
| I-6 | 320 | 90 |
| I-8 | 320 | 100 |
| I-10 | 320 | 90 |
| I-11 | 320 | 90 |
| I-14 | 1280 | 100 |
| I-41 | 1280 | 100 |
| I-43 | 1280 | 100 |
| I-45 | 1280 | 100 |
| I-46 | 1280 | 90 |
| I-178 | 1280 | 90 |
| I-179 | 1280 | 90 |
| I-201 | 1280 | 90 |
| I-244 | 1280 | 90 |
| I-245 | 1280 | 100 |
| I-277 | 1280 | 100 |
| I-278 | 1280 | 100 |
| I-288 | 1280 | 90 |
| I-289 | 1280 | 90 |
| I-291 | 1280 | 90 |
| I-292 | 1280 | 100 |
| I-294 | 1280 | 100 |
| I-295 | 1280 | 100 |
| I-296 | 1280 | 100 |

TABLE C7
Pre-emergence efficacy against Lolium rigidum (LOLRI)

| Example | Dosage | |
|---|---|---|
| number | [g/ha] | LOLRI |
| I-1 | 320 | 90 |
| I-2 | 320 | 90 |
| I-10 | 320 | 90 |
| I-294 | 320 | 100 |

TABLE C8
Pre-emergence efficacy against Matricaria inodora (MATIN)

| Example | Dosage | |
|---|---|---|
| number | [g/ha] | MATIN |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 100 |
| I-10 | 320 | 100 |
| I-11 | 320 | 100 |
| I-14 | 320 | 100 |
| I-41 | 320 | 100 |
| I-43 | 320 | 90 |
| I-45 | 320 | 100 |
| I-46 | 320 | 100 |
| I-178 | 320 | 100 |
| I-179 | 320 | 100 |
| I-180 | 320 | 100 |
| I-200 | 320 | 90 |
| I-245 | 320 | 90 |
| I-277 | 320 | 90 |
| I-292 | 320 | 100 |
| I-294 | 320 | 100 |
| I-295 | 320 | 100 |
| I-296 | 320 | 100 |
| I-297 | 320 | 100 |
| I-298 | 320 | 90 |

TABLE C9
Pre-emergence efficacy against Poa annua (POAAN)

| Example | Dosage | |
|---|---|---|
| number | [g/ha] | POAAN |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 100 |
| I-10 | 320 | 90 |
| I-11 | 320 | 100 |
| I-12 | 320 | 100 |
| I-14 | 320 | 100 |
| I-41 | 320 | 100 |
| I-43 | 320 | 100 |
| I-45 | 320 | 100 |
| I-178 | 320 | 100 |
| I-179 | 320 | 100 |
| I-180 | 320 | 100 |
| I-200 | 320 | 90 |
| I-201 | 320 | 100 |
| I-277 | 320 | 100 |
| I-288 | 320 | 90 |
| I-294 | 320 | 100 |
| I-295 | 320 | 90 |
| I-296 | 320 | 100 |
| I-299 | 320 | 90 |

TABLE C10
Pre-emergence efficacy against Setaria viridis (SETVI)

| Example | Dosage | |
|---|---|---|
| number | [g/ha] | SETVI |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 90 |
| I-10 | 320 | 90 |
| I-11 | 320 | 90 |
| I-14 | 320 | 100 |
| I-41 | 320 | 100 |
| I-43 | 320 | 100 |
| I-45 | 320 | 100 |
| I-178 | 320 | 100 |
| I-179 | 320 | 100 |
| I-277 | 320 | 100 |
| I-292 | 320 | 90 |
| I-294 | 320 | 100 |
| I-295 | 320 | 100 |
| I-296 | 320 | 100 |
| I-299 | 320 | 90 |

TABLE C11
Pre-emergence efficacy against Stellaria media (STEME)

| Example | Dosage | |
|---|---|---|
| number | [g/ha] | STEME |
| I-1 | 320 | 100 |
| I-2 | 320 | 100 |
| I-3 | 320 | 100 |
| I-5 | 320 | 100 |
| I-6 | 320 | 100 |
| I-7 | 320 | 100 |
| I-8 | 320 | 100 |
| I-10 | 320 | 100 |
| I-11 | 320 | 100 |
| I-12 | 320 | 90 |
| I-14 | 320 | 100 |
| I-41 | 320 | 100 |
| I-43 | 320 | 100 |
| I-45 | 320 | 100 |
| I-46 | 320 | 90 |
| I-178 | 320 | 100 |
| I-179 | 320 | 100 |
| I-180 | 320 | 100 |
| I-200 | 320 | 100 |
| I-201 | 320 | 90 |
| I-202 | 320 | 100 |
| I-245 | 320 | 100 |
| I-277 | 320 | 100 |
| I-278 | 320 | 100 |
| I-288 | 320 | 90 |
| I-289 | 320 | 90 |
| I-292 | 320 | 100 |
| I-294 | 320 | 100 |
| I-295 | 320 | 100 |
| I-296 | 320 | 100 |
| I-297 | 320 | 100 |
| I-298 | 320 | 100 |
| I-299 | 320 | 100 |
| I-300 | 320 | 90 |

TABLE C12
Pre-emergence efficacy against Veronica persica (VERPE)

| Example | Dosage | |
|---|---|---|
| number | [g/ha] | VERPE |
| I-1 | 320 | 100 |
| I-2 | 320 | 100 |
| I-3 | 320 | 100 |
| I-5 | 320 | 90 |
| I-6 | 320 | 100 |
| I-7 | 320 | 100 |
| I-8 | 320 | 100 |
| I-10 | 320 | 100 |
| I-11 | 320 | 100 |
| I-41 | 320 | 100 |
| I-43 | 320 | 100 |
| I-45 | 320 | 100 |
| I-178 | 320 | 100 |
| I-179 | 320 | 90 |
| I-244 | 320 | 90 |
| I-277 | 320 | 100 |
| I-278 | 320 | 100 |
| I-292 | 320 | 90 |
| I-294 | 320 | 100 |
| I-295 | 320 | 90 |
| I-296 | 320 | 90 |

As shown by way of example by the results in tables C1-C12, inventive compounds No. I-1, I-2, I-3, I-5

, I-6, I-7, I-8, I-10, I-11, I-12, I-14, I-41, I-43, I-45, I-46, I-178, I-179, I-180, I-200, I-201, I-202, I-245, I-277, I-278, I-288, I-289, I-291, I-292, I-294, I-295, I-296, I-297,

I-298, I-299 and I-300 in the case of pre-emergence treatment have very good herbicidal efficacy against the following harmful plants at an application rate of 320 g of active substance per hectare: Abutilon theophrasti (ABUTH), Alopecurus myosuroides (ALOMY), Amaranthus retroflexus (AMARE), Digitaria sanguinalis (DIGSA), Echinochloa crus-galli (ECHCG), Bassia scoparia (KCHSC), Lolium rigidum (LOLRI), Matricaria inodora (MATIN), Poa annua (POAAN), Setaria viridis (SETVI), Stellaria media (STEME) and Veronica persica (VERPE).

### D. Pre-Emergence Herbicide Efficacy at 80 g/ha

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate of 6001/ha (converted). After the treatment, the pots were placed in a greenhouse and kept under good growth conditions for the test plants. After about 3 weeks, the effect of the preparations was scored visually in comparison with untreated controls as percentages. For example, 100% activity=the plants have died, 0% activity=like control plants.

Tables D1 to D12 below show the effects of selected compounds of the general formula (I) according to Table 1 on various harmful plants and an application rate corresponding to 80 g/ha, which were obtained by the experimental procedure mentioned above.

TABLE D1
Pre-emergence efficacy against Abutilon theophrasti (ABUTH)

| Example | Dosage | |
|---|---|---|
| number | [g/ha] | ABUTH |
| I-1 | 80 | 80 |
| I-2 | 80 | 90 |
| I-4 | 80 | 100 |
| I-5 | 80 | 90 |
| I-7 | 80 | 100 |
| I-8 | 80 | 100 |
| I-10 | 80 | 90 |
| I-14 | 80 | 80 |
| I-41 | 80 | 80 |
| I-45 | 80 | 90 |
| I-277 | 80 | 90 |
| I-293 | 80 | 90 |
| I-294 | 80 | 90 |

TABLE D2
Pre-emergence efficacy against Alopecurus myosuroides (ALOMY)

| Example | Dosage | |
|---|---|---|
| number | [g/ha] | ALOMY |
| I-1 | 80 | 90 |
| I-2 | 80 | 80 |
| I-4 | 80 | 90 |
| I-5 | 80 | 90 |
| I-7 | 80 | 100 |
| I-8 | 80 | 90 |
| I-41 | 80 | 80 |
| I-277 | 80 | 80 |
| I-292 | 80 | 80 |
| I-293 | 80 | 90 |
| I-294 | 80 | 90 |

TABLE D3
Pre-emergence efficacy against Amaranthus retroflexus (AMARE)

| Example | Dosage | |
|---|---|---|
| number | [g/ha] | AMARE |
| I-1 | 80 | 100 |
| I-2 | 80 | 100 |
| I-3 | 80 | 100 |
| I-4 | 80 | 100 |
| I-5 | 80 | 100 |
| I-7 | 80 | 100 |
| I-8 | 80 | 100 |
| I-10 | 80 | 90 |
| I-14 | 80 | 100 |
| I-41 | 80 | 100 |
| I-43 | 80 | 100 |
| I-45 | 80 | 100 |
| I-277 | 80 | 100 |
| I-292 | 80 | 100 |
| I-293 | 80 | 100 |
| I-294 | 80 | 100 |

TABLE D4
Pre-emergence efficacy against Avena fatua (AVEFA)

| Example | Dosage | |
|---|---|---|
| number | [g/ha] | AVEFA |
| I-4 | 80 | 80 |

TABLE D5
Pre-emergence efficacy against Digitaria sanguinalis (DIGSA)

| Example | Dosage | |
|---|---|---|
| number | [g/ha] | DIGSA |
| I-3 | 80 | 100 |
| I-4 | 80 | 100 |
| I-10 | 80 | 90 |

TABLE D6
Pre-emergence efficacy against Matricaria inodora (MATIN)

| Example | Dosage | |
|---|---|---|
| number | [g/ha] | MATIN |
| I-1 | 80 | 100 |
| I-3 | 80 | 100 |
| I-4 | 80 | 90 |
| I-5 | 80 | 90 |
| I-7 | 80 | 90 |
| I-10 | 80 | 90 |
| I-41 | 80 | 80 |
| I-43 | 80 | 90 |
| I-277 | 80 | 100 |
| I-293 | 80 | 100 |
| I-294 | 80 | 100 |

TABLE D7
Pre-emergence efficacy against Ipomoea purpurea (PHBPU)

| Example | Dosage | |
|---|---|---|
| number | [g/ha] | PHBPU |
| I-4 | 80 | 80 |
| I-277 | 80 | 100 |
| I-294 | 80 | 100 |

TABLE D8
Pre-emergence efficacy against Polygonum convolvulus (POLCO)

| Example | Dosage | |
|---|---|---|
| number | [g/ha] | POLCO |
| I-1 | 80 | 90 |
| I-2 | 80 | 80 |
| I-3 | 80 | 90 |
| I-4 | 80 | 90 |
| I-5 | 80 | 90 |
| I-7 | 80 | 90 |
| I-8 | 80 | 90 |
| I-10 | 80 | 90 |
| I-41 | 80 | 90 |
| I-45 | 80 | 80 |
| I-277 | 80 | 100 |
| I-293 | 80 | 80 |
| I-294 | 80 | 90 |

TABLE D9
Pre-emergence efficacy against Setaria viridis (SETVI)

| Example | Dosage | |
|---|---|---|
| number | [g/ha] | SETVI |
| I-1 | 80 | 80 |
| I-4 | 80 | 100 |
| I-5 | 80 | 90 |
| I-7 | 80 | 100 |
| I-8 | 80 | 100 |
| I-41 | 80 | 80 |
| I-43 | 80 | 90 |
| I-45 | 80 | 90 |
| I-277 | 80 | 100 |
| I-293 | 80 | 100 |
| I-294 | 80 | 100 |

TABLE D10
Pre-emergence efficacy against Veronica persica (VERPE)

| Example | Dosage | |
|---|---|---|
| number | [g/ha] | VERPE |
| I-1 | 80 | 90 |
| I-2 | 80 | 90 |
| I-3 | 80 | 80 |
| I-4 | 80 | 100 |
| I-5 | 80 | 90 |
| I-7 | 80 | 90 |
| I-8 | 80 | 80 |
| I-41 | 80 | 80 |
| I-43 | 80 | 90 |
| I-45 | 80 | 80 |
| I-277 | 80 | 80 |
| I-292 | 80 | 90 |
| I-293 | 80 | 80 |

TABLE D11
Pre-emergence efficacy against Viola tricolor (VIOTR)

| Example | Dosage | |
|---|---|---|
| number | [g/ha] | VIOTR |
| I-1 | 80 | 90 |
| I-2 | 80 | 90 |
| I-3 | 80 | 100 |
| I-4 | 80 | 100 |
| I-5 | 80 | 90 |
| I-7 | 80 | 100 |
| I-8 | 80 | 90 |
| I-10 | 80 | 100 |
| I-14 | 80 | 100 |
| I-41 | 80 | 80 |
| I-43 | 80 | 80 |
| I-45 | 80 | 90 |
| I-277 | 80 | 100 |
| I-292 | 80 | 90 |
| I-293 | 80 | 90 |
| I-294 | 80 | 100 |

TABLE D12
Pre-emergence efficacy against Echinochloa crus-galli (ECHCG)

| Example | Dosage | |
|---|---|---|
| number | [g/ha] | ECHCG |
| I-45 | 80 | 80 |
| I-277 | 80 | 100 |
| I-294 | 80 | 90 |

As shown by way of example by the results in tables D1-D12, inventive compounds No. I-1, I-2, I-3, I-4, I-5, I-7, I-8, I-10, I-14, I-41, I-43, I-45, I-277, I-292, I-293 and I-294 in the case of pre-emergence treatment have very good herbicidal efficacy against the following harmful plants at an application rate of 80 g of active substance per hectare: Abutilon theophrasti (ABUTH), Alopecurus myosuroides (ALOMY), Amaranthus retroflexus (AMARE), Avena fatua (AVEFA), Digitaria sanguinalis (DIGSA), Echinochloa crus-galli (ECHCG), Matricaria inodora (MATIN), Ipomoea purpurea (PHBPU), Polygonum convolvulus (POLCO), Setaria viridis (SETVI), Veronica persica (VERPE) and Viola tricolor (VIOTR).

1. A substituted heteroaryloxypyridine of formula (I) or salt thereof 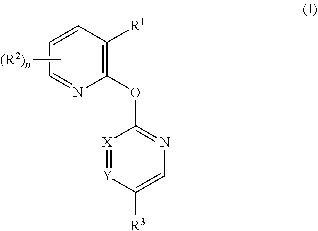
in which
R¹ is an optionally substituted 5-membered heteroaryl ring optionally substituted by up to 3 substituents independently selected from the group of R⁴,
R² is independently halogen, hydroxy, amino, cyano, nitro, formyl, formamide, (C₁-C₄)-alkyl, (C₁-C₄)-haloalkyl, (C₃-C₆)-cycloalkyl, (C₂-C₄)-alkenyl, (C₂-C₄)-alkynyl, (C₂-C₄)-haloalkenyl, (C₂-C₄)-haloalkynyl, (C₁-C₄)-alkoxy, (C₁-C₄)-haloalkoxy, (C₃-C₆)-cycloalkoxy, (C₁-C₄)-alkenyloxy, (C₁-C₄)-alkynyloxy, (C₁-C₄)-alkylthio, (C₁-C₄)-haloalkylthio, (C₃-C₆)-cycloalkylthio, (C₁-C₄)-alkylsulfinyl, (C₁-C₄)-haloalkylsulfinyl, (C₃-C₆)-cycloalkylsulfinyl, (C₁-C₄)-alkylsulfonyl, (C₁-C₄)-haloalkylsulfonyl, (C₃-C₆)-cycloalkylsulfonyl, (C₁-C₄)-alkoxy-(C₁-C₄)-alkyl, (C₁-C₄)-haloalkoxy-(C₁-C₄)-alkyl, (C₁-C₄)-alkylthio-(C₁-C₄)-alkyl, (C₁-C₄)-alkylsulfinyl-(C₁-C₄)-alkyl, (C₁-C₄)-alkylsulfonyl-(C₁-C₄)-alkyl, (C₁-C₄)-alkylcarbonyl, (C₁-C₄)-haloalkylcarbonyl, (C₃-C₆)-cycloalkylcarbonyl, carboxyl, (C₁-C₄)-alkoxycarbonyl, (C₁-C₄)-haloalkoxycarbonyl, (C₃-C₆)-cycloalkoxycarbonyl, (C₁-C₄)-alkylaminocarbonyl, (C₂-C₆)-dialkylaminocarbonyl, (C₃-C₆)-cycloalkylaminocarbonyl, (C₁-C₄)-alkylcarbonylamino, (C₁-C₄)-haloalkylcarbonylamino, (C₂-C₆)-cycloalkylcarbonylamino, (C₁-C₄)-alkoxycarbonylamino, (C₁-C₄)-alkylaminocarbonylamino, (C₂-C₆)-dialkylaminocarbonylamino, carboxy-(C₁-C₄)-alkyl, (C₁-C₄)-alkoxycarbonyl-(C₁-C₄)-alkyl, (C₁-C₄)-haloalkoxycarbonyl-(C₁-C₄)-alkyl, (C₃-C₆)-cycloalkoxycarbonyl-(C₁-C₄)-alkyl, (C₁-C₄)-alkylaminosulfonyl, (C₂-C₆)- dialkylaminosulfonyl or (C₃-C₆)-trialkylsilyl,
n is 0, 1, 2 or 3,
R³ is halogen, cyano, nitro, (C₁-C₄)-alkyl or (C₁-C₄)-haloalkyl,
R⁴ is hydrogen, halogen, hydroxy, amino, cyano, formyl, (C₁-C₄)-alkyl, (C₁-C₄)-haloalkyl, (C₃-C₆)-cycloalkyl, (C₁-C₄)-alkoxy, (C₁-C₄)-haloalkoxy, (C₁-C₄)-alkoxycarbonyl, (C₁-C₄)-alkoxythiocarbonyl, (C₃-C₆)-cycloalkyl-(C₁-C₄)-alkyl, (C₁-C₄)-alkyl-(C₃-C₆)-cycloalkyl or (C₁-C₄)-alkoxy-(C₁-C₄)-alkyl,
X is N or CR⁵,
Y is N or CH,
and
R⁵ is hydrogen, halogen or cyano,
excluding compounds in which R¹ is an unsubstituted or substituted 3-pyrazole.

2. The compound of formula (I) as claimed in claim 1 or salt thereof, in which
R¹ is the groups R¹-1 to R¹-42: 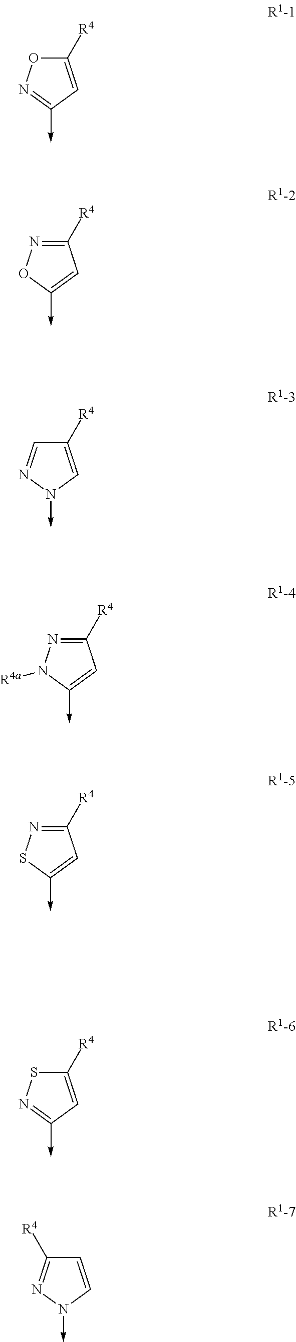 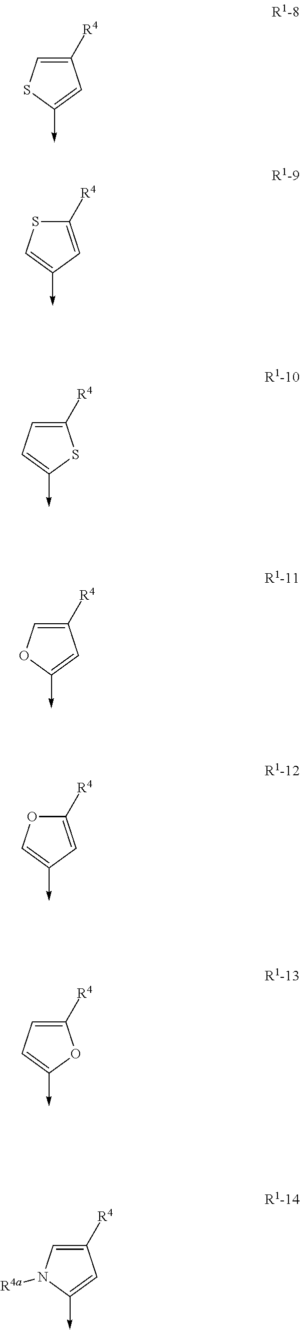 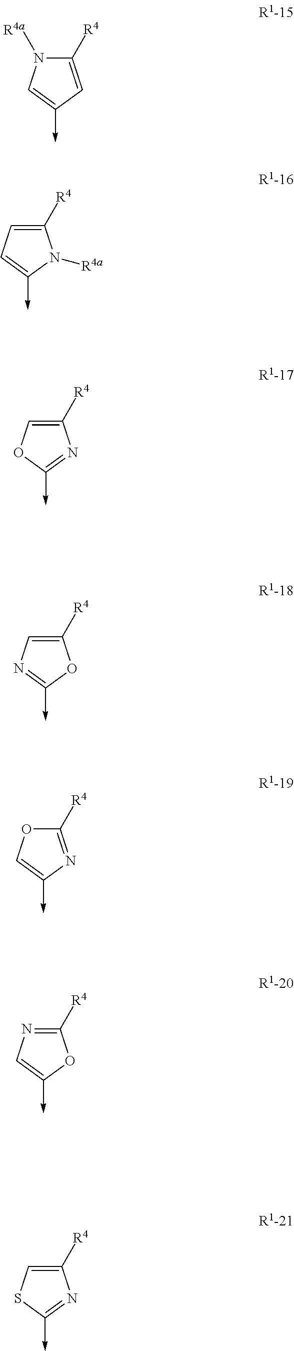 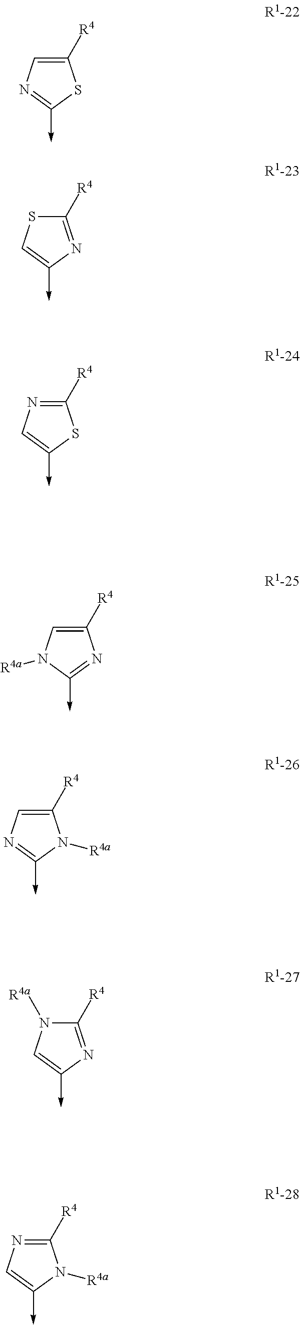 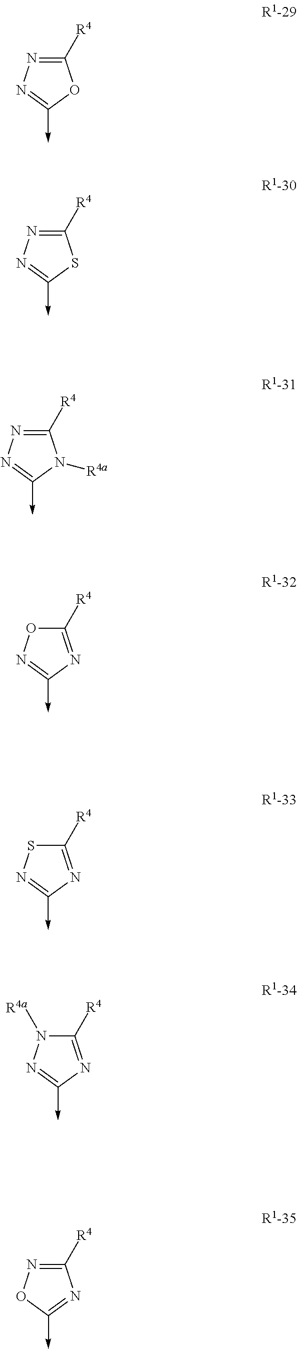 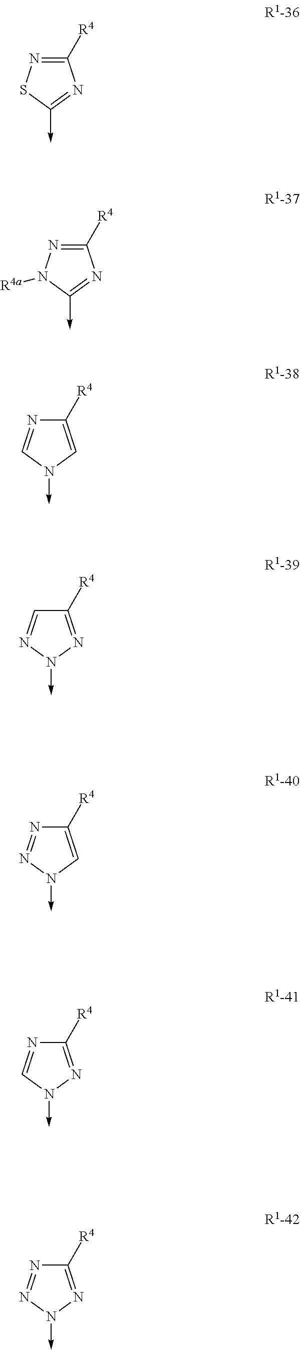
R² is independently halogen, hydroxy, amino, cyano, nitro, formyl, formamide, (C₁-C₄)-alkyl, (C₁-C₄)-haloalkyl, (C₃-C₆)-cycloalkyl, (C₂-C₄)-alkenyl, (C₂-C₄)-alkynyl, (C₂-C₄)-haloalkenyl, (C₂-C₄)-haloalkynyl, (C₁-C₄)-alkoxy, (C₁-C₄)-haloalkoxy, (C₃-C₆)-cycloalkoxy, (C₁-C₄)-alkenyloxy, (C₁-C₄)-alkynyloxy, (C₁-C₄)-alkylthio, (C₁-C₄)-haloalkylthio, (C₃-C₆)-cycloalkylthio, (C₁-C₄)-alkylsulfinyl, (C₁-C₄)-haloalkylsulfinyl, (C₃-C₆)-cycloalkylsulfinyl, (C₁-C₄)-alkylsulfonyl, (C₁-C₄)-haloalkylsulfonyl, (C₃-C₆)-cycloalkylsulfonyl, (C₁-C₄)-alkoxy-(C₁-C₄)-alkyl, (C₁-C₄)-haloalkoxy-(C₁-C₄)-alkyl, (C₁-C₄)- alkylcarbonyl, (C₁-C₄)-haloalkylcarbonyl, (C₃-C₆)-cycloalkylcarbonyl, carboxyl, (C₁-C₄)-alkoxycarbonyl, (C₁-C₄)-haloalkoxycarbonyl, (C₃-C₆)-cycloalkoxycarbonyl, (C₁-C₄)-alkylaminocarbonyl, (C₂-C₆)- dialkylaminocarbonyl, (C₃-C₆)-cycloalkylaminocarbonyl, (C₁-C₄)-alkylcarbonylamino, (C₁-C₄)-haloalkylcarbonylamino, (C₂-C₆)-cycloalkylcarbonylamino, (C₁-C₄)-alkoxycarbonylamino or (C₃-C₆)-trialkylsilyl,
n is 0, 1 or 2,
R³ is halogen, cyano, nitro, (C₁-C₄)-alkyl or (C₁-C₄)-haloalkyl,
R⁴ is hydrogen, halogen, hydroxy, amino, cyano, formyl, (C₁-C₄)-alkyl, (C₁-C₄)-haloalkyl, (C₃-C₆)-cycloalkyl, (C₁-C₄)-alkoxy, (C₁-C₄)-haloalkoxy, (C₁-C₄)-alkoxycarbonyl, (C₁-C₄)-alkoxythiocarbonyl, (C₃-C₆)-cycloalkyl-(C₁-C₄)-alkyl, (C₁-C₄)-alkyl-(C₃-C₆)-cycloalkyl or (C₁-C₄)-alkoxy-(C₁-C₄)-alkyl,
R^{4a} is hydrogen or (C₁-C₂)-alkyl,
X is N or CR⁵,
Y is N or CH,
and
R⁵ is hydrogen, halogen or cyano.

3. The compound of the general formula (I) as claimed in claim 1 or salt thereof, in which
R¹ is the groups R¹-1 to R¹ 3, 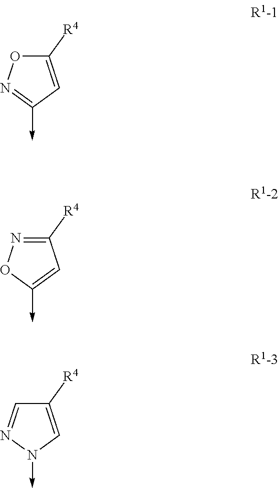
R2 is independently chlorine, bromine, cyano or methyl,
n is 1 or 2,
R³ is fluorine, chlorine, bromine, cyano, nitro or trifluoromethyl,
R⁴ is hydrogen, chlorine, bromine, iodine, trifluoromethyl, difluoromethyl, chlorofluoromethyl, difluorochloromethyl, dichloromethyl, trichloromethyl, difluorobromomethyl, cyclopropyl, cyclopropylmethyl, (1-methyl)cyclopropyl or methoxymethyl,
X is N or CR⁵,
Y is N or CH,
and
R⁵ is hydrogen, fluorine, chlorine or cyano.

4. A herbicidal composition comprising a herbicidally active amount of at least one compound of formula (I) and/or salt thereof as claimed in claim 1.

5. The herbicidal composition as claimed in claim 4 in a mixture with one or more formulation auxiliaries.

6. The herbicidal composition as claimed in claim 4, comprising at least one further pesticidally active substance from the group of insecticides, acaricides, herbicides, fungicides, safeners, and growth regulators.

7. The herbicidal composition as claimed in claim 6, comprising a safener.

8. The herbicidal composition as claimed in claim 7, comprising cyprosulfamide, cloquintocet-mexyl, mefenpyr-diethyl or isoxadifen-ethyl.

9. The herbicidal composition as claimed in claim 4, comprising a further herbicide.

10. A method of controlling one or more unwanted plants, comprising applying an effective amount of at least one compound of formula (I) as claimed in claim 1 or of a herbicidal composition thereof to the plants or to a site of unwanted vegetation.

11. The use of A product comprising a compound of formula (I) and/or salt as claimed in claim 1 or herbicidal composition thereof for controlling one or more unwanted plants.

12. The product use as claimed in claim 11, wherein the compound of formula (I) and/or salt is adapted for use for controlling one or more unwanted plants in one or more crops of one or more useful plants.

13. The product as claimed in claim 12, wherein the useful plants are transgenic useful plants.
